(19) United States
(12) Patent Application Publication
(10) Pub. No.: US 2026/0283485 A1
(43) Pub. Date: Sep. 24, 2026

(54) SENSOR-FUSION-BASED BLOOD PRESSURE MEASUREMENT

(71) Applicant: Google LLC, Mountain View, CA (US)

(72) Inventors: Rajeev Nongpiur, Mountain View, CA (US); Nina Sinatra, Mountain View, CA (US); Luzhou Xu, Mountain View, CA (US); Jaime Lien, Mountain View, CA (US); Qian Zhang, Mountain View, CA (US); Jihan Li, Mountain View, CA (US)

(73) Assignee: Google LLC, Mountain View, CA (US)

(21) Appl. No.: 19/477,617

(22) PCT Filed: Apr. 28, 2023
(86) PCT No.: PCT/US2023/020406

§ 371 Date: Oct. 22, 2025

(51) Int. Cl. A61B 5/021; A61B 5/00; A61B 5/024; A61B 5/05

(52) U.S. Cl. CPC A61B 5/02125; A61B 5/0004; A61B 5/02416; A61B 5/05; A61B 5/681; A61B 5/7278

(57) ABSTRACT

Various arrangements for measuring blood pressure using sensor fusion are presented herein. Radio frequency (RF) signals are emitted and RF reflection signals are received by a radar sensor of a stationary device. The RF reflection signals are analyzed at a first distance range to identify a first pulse pressure waveform (PPW) at an aortic valve of a user. Vital sign data measured at an extremity of the user by a mobile device are received by the stationary device. The vital sign data is analyzed to identify a second PPW at the extremity of the user. Using the first PPW and the second PPW, a pulse transit time from the aortic valve to the extremity is determined. Using the PTT, a blood pressure (BP) of the user is determined and an indication of the BP is output.

### BACKGROUND

Blood pressure (BP) is an important vital statistic for measuring health. A person may desire to occasionally or periodically measure their BP to ensure it is within healthy limits. A sudden decrease or increase in BP can serve as a warning that a person should seek medical help. Typically, BP measurement devices for at home use are inconvenient and uncomfortable. Some require that a high level of pressure be applied by a cuff to the person's arm. Others require that a specialized device be clipped to a finger or other extremity. In still other arrangements, multiple specialized sensors may be used in a hybrid approach that combines an electrocardiogram (ECG) with a photoplethysmography (PPG) sensor, which can result in an unreliable BP measurement.

Such arrangements are not conducive to convenient and consistent personal BP monitoring. First, such arrangements require specialized devices that need to be stored and retrieved each time a person desires to measure their BP. Second, such devices may be prone to user error, resulting in inaccurate BP measurements. Embodiments described herein can address these and other issues.

### SUMMARY

In some embodiments, a blood pressure measurement system is presented. The system can include a mobile device wearable by a user and configured to collect vital sign data at an extremity of the user and a stationary device in communication with the mobile device. The stationary device can include a radio frequency (RF) emitter that emits RF signals, an RF receiver that receives RF reflection signals based on the emitted RF signals being reflected, and one or more processors. The one or more processors can be configured to analyze the RF reflection signals at a first distance range to identify a first pulse pressure waveform at an aortic valve of the user. The one or more processors can be further configured to analyze the vital sign data to identify a second pulse pressure waveform at the extremity of the user. The one or more processors can be further configured to determine a pulse transit time (PTT) of a pulse pressure wave from the aortic valve of the user to the extremity of the user using the first pulse pressure waveform and the second pulse pressure waveform. The one or more processors can be further configured to determine a blood pressure of the user based on the determined PTT. The one or more processors can be further configured to output an indication of the determined blood pressure.

In some embodiments, the vital sign data comprises photoplethysmography (PPG) data and the mobile device comprises an optical sensor that measures the PPG data at the extremity of the user. In some embodiments, the stationary device further comprises: a microphone, a speaker, and an electronic display, wherein the indication of the determined blood pressure is output via the electronic display. In some embodiments, the mobile device is a smart watch and the extremity of the user is a wrist of the user. The smart watch may further comprise: a microphone, a speaker, and an electronic display, wherein the indication of the determined blood pressure is output via the electronic display.

In some embodiments, the RF reflection signals are associated with a first set of timestamps generated by a first clock of the stationary device, the vital sign data is associated with a second set of timestamps generated by a second clock of the mobile device, and the one or more processors are further configured to synchronize the first set of timestamps with the second set of timestamps and adjust the first set of timestamps, the second set of timestamps, or both based on a processing-delay difference between the stationary device and the mobile device. In some embodiments, the one or more processors are further configured to receive an external blood pressure measurement made using a blood pressure device separate from the stationary device and the mobile device, compare the external blood pressure measurement and the determined blood pressure measurement, and create a calibration profile for use in modifying a future determined blood pressure measurement. In some embodiments, the one or more processors are further configured to use the RF reflection signals and the vital sign data as inputs to a machine learning model trained to derive the blood pressure of the user based on the PTT.

In some embodiments, a method for measuring blood pressure is presented. The method may include emitting, by a radar sensor of a stationary device, radio frequency (RF) signals. The method may further include receiving, by the radar sensor of the stationary device, RF reflection signals based on the emitted RF signals being reflected. The method may further include analyzing, by a processing system of the stationary device, the RF reflection signals at a first distance range to identify a first pulse pressure waveform at an aortic valve of a user. The method may further include receiving, by the processing system of the stationary device, vital sign data measured at an extremity of the user by a mobile device. The method may further include analyzing, by the processing system of the stationary device, the vital sign data to identify a second pulse pressure waveform at the extremity of the user. The method may further include determining, by the processing system of the stationary device, a pulse transit time (PTT) of a pulse pressure wave from the aortic valve of the user to the extremity of the user using the first pulse pressure waveform and the second pulse pressure waveform. The method may further include determining, by the processing system of the stationary device, a blood pressure of the user based on the determined PTT. The method may further include outputting, by the processing system of the stationary device, an indication of the determined blood pressure.

In some embodiments, the vital sign data comprises photoplethysmography (PPG) data, and the method further comprises measuring, by an optical sensor of the mobile device, the PPG data at the extremity of the user. In some embodiments, the RF reflection signals are associated with a first set of timestamps generated by a first clock of the stationary device, the vital sign data is associated with a second set of timestamps generated by a second clock of the mobile device, and the method further comprises synchronizing the first set of timestamps with the second set of timestamps. Embodiments of such a method may further include adjusting the first set of timestamps, the second set of timestamps, or both, based on a processing-delay difference between the stationary device and the mobile device.

The method may further include determining a phase difference between the first pulse pressure waveform and the second pulse pressure waveform, wherein the PTT is determined using the phase difference. In some embodiments, the extremity of the user is a wrist of the user. In some embodiments, the method further includes determining a heart rate based on analyzing the RF reflection signals, the second pulse pressure waveform, or both, wherein determining the blood pressure of the user is further based on the heart rate. In some embodiments, determining a derived pulse waveform amplitude (DPWA) based on analyzing the RF reflection signals, wherein determining the blood pressure of the user is further based on the DPWA. In some embodiments, determining a respiration rate based on analyzing the RF reflection signals, the second pulse pressure waveform, or both, wherein determining the blood pressure of the user is further based on the respiration rate.

In some embodiments, analyzing the RF reflection signals at the first distance range comprises analyzing data from the RF reflection signals using a trained machine learning model. In some embodiments, the method further includes receiving, by the processing system of the stationary device, an external blood pressure measurement made using a blood pressure device separate from the stationary device and the mobile device, comparing, by the processing system, the external blood pressure measurement and the determined blood pressure measurement, and creating, by the processing system, a calibration profile for use in modifying a future determined blood pressure measurement.

In some embodiments, a stationary blood pressure measurement device is presented. The device may include a radar subsystem. The radar subsystem can include a radio frequency (RF) emitter that emits RF signals, an RF receiver that receives RF reflection signals based on the emitted RF signals being reflected, and a processing system. The processing system can include one or more processors in communication with the radar subsystem. The processing system can be configured to analyze the RF reflection signals at a first distance range to identify a first pulse pressure waveform at an aortic valve of a user. The processing system can be further configured to receive vital sign data measured at an extremity of the user by a mobile device. The processing system can be further configured to analyze the vital sign data to identify a second pulse pressure waveform at the extremity of the user. The processing system can be further configured to determine a pulse transit time (PTT) of a pulse pressure wave from the aortic valve of the user to the extremity of the user using the first pulse pressure waveform and the second pulse pressure waveform. The processing system can be further configured to determine a blood pressure of the user based on the determined PTT. The processing system can be further configured to output an indication of the determined blood pressure.

### BRIEF DESCRIPTION OF THE DRAWINGS

A further understanding of the nature and advantages of various embodiments may be realized by reference to the following figures. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 illustrates an embodiment of a distributed BP measurement system that can be used to perform sensor-fusion-based BP measurements.

FIG. 2 illustrates a block diagram of an embodiment of a sensor-fusion-based BP measurement system.

FIG. 3 illustrates a block diagram of another embodiment of a sensor-fusion-based BP measurement system.

FIG. 4 illustrates an embodiment of frequency-modulated continuous wave radar radio waves output by a radar subsystem.

FIG. 5 illustrates an embodiment of a pulse wave measured by a distributed BP measurement system.

FIG. 6 illustrates an embodiment of a BP measurement device.

FIG. 7 illustrates an exploded view of an embodiment of a BP measurement device.

FIG. 8 illustrates an embodiment of a user having the user's BP measured using a distributed BP measurement system.

FIG. 9 illustrates another embodiment of a user having their BP measured using a distributed BP measurement system.

FIG. 10 illustrates an embodiment of a method for measuring BP.

FIG. 11 illustrates another embodiment of a method for measuring BP.

FIG. 12 illustrates an embodiment of a method for calibrating a distributed BP measurement system.

### DETAILED DESCRIPTION

Pulse transit time (PTT) can refer to an amount of time taken by a pulse pressure wave (PPW) to travel between two arterial sites, such as from a person's aortic value to the person's extremities (e.g., wrist, finger, foot, etc.). The speed at which a PPW travels through a person's vasculature, and therefore the PTT, is proportional to BP. As the BP of a person increases, speed, and therefore the PTT, decreases. Accordingly, by determining a PTT for a person, possibly in conjunction with other measurements, the BP for the person can be determined.

Embodiments detailed herein are focused on embodiments of a sensor-fusion-based BP measurement system and associated methods that allow for BP measurement using distributed sensing devices. A sensor-fusion-based BP measurement system can include a stationary device equipped with a radar sensor that can detect small movements at discrete distances. Therefore, movement at a person's aortic valve corresponding to PPWs generated by the person's heart can be distinguished from other movement in the surrounding environment. The sensor-fusion-based BP measurement system can also include a mobile device equipped with an optical sensor that can detect PPG data from a user. When placed on an extremity of the person, the PPG data can be used to detect the same PPWs once they have propagated from the aortic valve to the extremity. Using the PPWs from each device, the PTT from the aortic valve to the extremity of the user can be determined. Subsequently, the PTT from the aortic valve to the extremity can be used to determine a BP for the user.

As detailed herein, additional measurements can be performed using data from either device to determine a more accurate BP of a user, such as PPW amplitude (PPWA), heart rate (HR), respiration rate (RR), and the like. In some embodiments, as detailed herein, a trained machine learning model is used to either partially or fully analyze data obtained from a radar sensor and an optical sensor.

Components of such a distributed BP measurement system can be incorporated as part of a device that includes a radar sensor, such as a home assistant device. A home assistant device can present information to the user about their measured BP and may be in a physical position that lends itself to easy BP measurement. Use of such a form of device can be highly beneficial since it may typically be displayed in a prominent position within a person's home, such as on a table or bedstand, and can provide an easy opportunity for a user to conduct a BP measurement. Furthermore, components of such a system can be incorporated as part of a device that includes an optical sensor, such as a smart wearable device. A smart wearable device, such as a smart watch, can provide an additional layer of convenience by being readily accessible as long as a user is wearing the device.

BP measurements, as detailed herein, can only be performed with explicit approval of the user. The user may need to remain in a specific physical position and be relatively motionless in order for the BP measurement to be performed. During such a time, visual and/or auditory messages can be output indicating that the BP of the user is being measured. Further, the user may be required to provide a consent allowing the user's BP to be measured. The user may retain the ability to delete any BP measurement made using such a system.

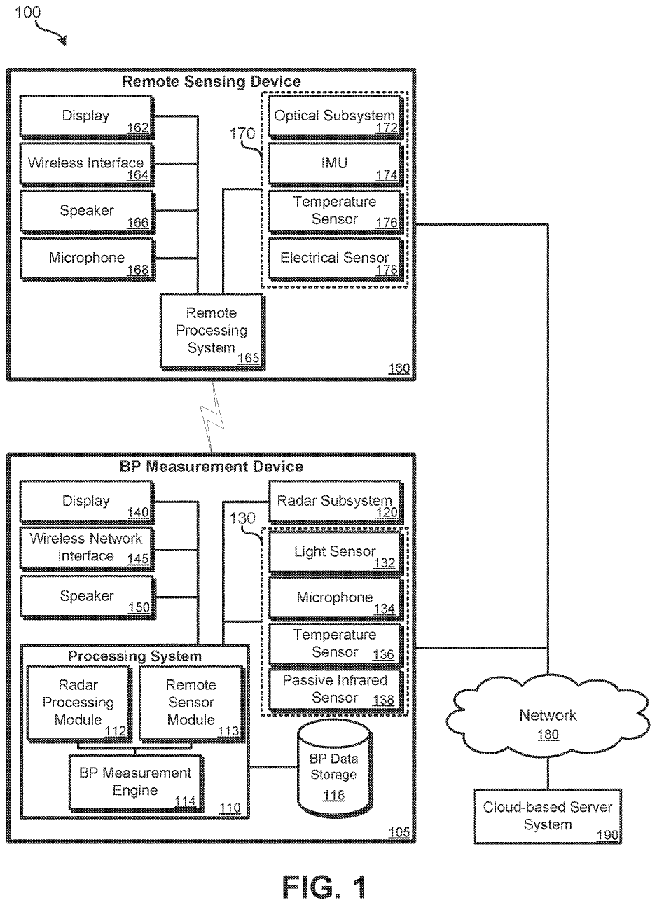

Further detail of these and other embodiments is provided in relation to the figures. FIG. 1 illustrates an embodiment of distributed BP measurement system 100 (“system 100”) that can be used to performed BP measurements. System 100 can include: BP measurement device 105; remote sensing device 160; network 180; and cloud-based server system 190. BP measurement device 105 may generally be a smart home assistant device, a smartphone, a smartwatch, laptop, gaming device, or a smart home hub device that is used to interact with various smart home devices present within a home. BP measurement device 105 can include: processing system 110; BP data storage 118; radar subsystem 120; environmental sensor suite 130; display 140; wireless network interface 145; and speaker 150. Generally, BP measurement device 105 can include a housing that houses all of the components of BP measurement device 105.

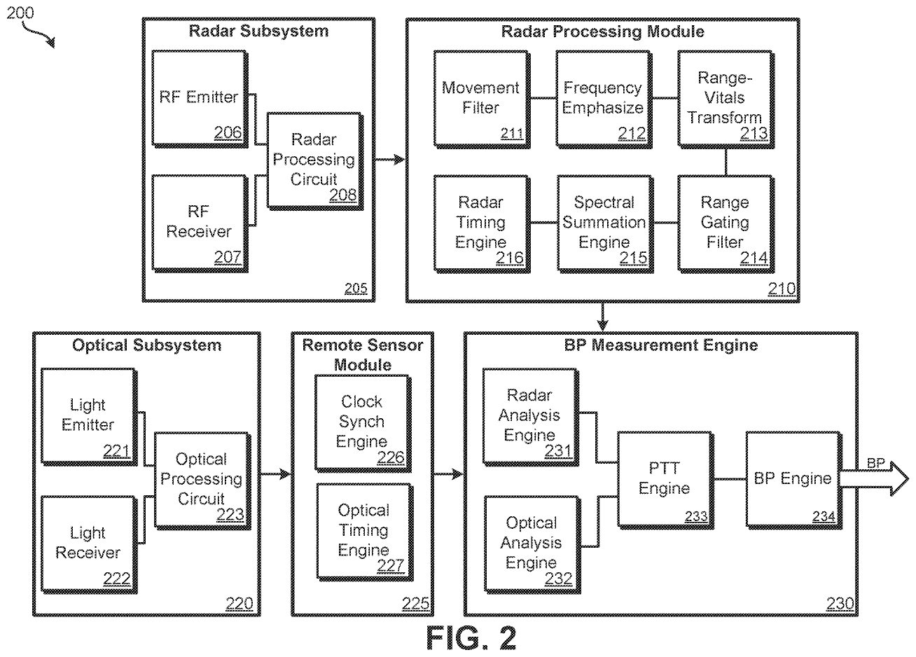

Processing system 110 can include one or more processors configured to perform various functions, such as the functions of: radar processing module 112; remote sensor module 113; and BP measurement engine 114. As described further herein, radar processing module 112 and remote sensor module 113 can analyze raw or otherwise unprocessed radar data and remote sensor data, respectively, to provide inputs for BP measurement engine 114. For example, radar processing module 112 can receive data from radar subsystem 120. Radar subsystem 120 (also referred to as a radar sensor) can be a single integrated circuit (IC) that emits, receives, and outputs data indicative of a received, reflected waveform. Radar processing module 112 can then process the reflected waveform to identify micromotions at different distances from BP measurement device 105. As another example, remote sensor module 113 can process vital sign data from remote sensing device 160. BP measurement engine 114 may subsequently receive the processed data from radar processing module 112 and/or remote sensor module 113 to derive measurements indicative of a user's BP. Further detail regarding radar subsystem 120, radar processing module 112, remote sensor module 113, and BP measurement engine 114 is provided in relation to FIGS. 2-4.

Processing system 110 can include one or more special-purpose or general-purpose processors. Such special-purpose processors may include processors that are specifically designed to perform the functions detailed herein. Such special-purpose processors may be ASICs or FPGAs which are general-purpose components that are physically and electrically configured to perform the functions detailed herein. Such general-purpose processors may execute special-purpose software that is stored using one or more non-transitory processor-readable mediums, such as random access memory (RAM), flash memory, a hard disk drive (HDD), or a solid state drive (SSD).

BP measurement device 105 may include one or more environmental sensors, such as all, one, or some combination of the environmental sensors provided as part of environmental sensor suite 130. Environmental sensor suite 130 can include: light sensor 132; microphone 134; temperature sensor 136; and passive infrared (PIR) sensor 138. In some embodiments, multiple instances of some or all of these sensors may be present. For instance, in some embodiments, multiple microphones may be present. Light sensor 132 may be used for measuring an ambient amount of light present in the general environment of BP measurement device 105. Microphone 134 may be used for measuring an ambient noise level present in the general environment of BP measurement device 105. Temperature sensor 136 may be used for measuring an ambient temperature of the general environment of BP measurement device 105. PIR sensor 138 may be used to detect moving living objects (e.g., persons, pets) within the general environment of BP measurement device 105.

Other types of environmental sensors are possible. For instance, a camera and/or humidity sensor may be incorporated as part of environmental sensor suite 130. As another example, active infrared sensors may be included. In some embodiments, some data, such as humidity data, may be obtained from a nearby weather station that has data available via the Internet. In some embodiments, active acoustic sensing methods, including, but not limited to, sonar and ultrasound, and including either single or arrayed acoustic sources and/or receivers may be implemented. Such arrangements may be used as one or more adjunct sensing modalities incorporated with the other sensors and methods described herein.

In some embodiments, one, some, or all sensors of environmental sensor suite 130 may be external to BP measurement device 105. For instance, one or more remote environmental sensors may communicate with BP measurement device 105, either directly (e.g., via a direct wireless communication method, via a low-power mesh network) or indirectly (e.g., through one or more other devices via the low-power mesh network, via an access point of a network, via a remote server).

BP measurement device 105 may include various interfaces, such as display 140, wireless network interface 145, and speaker 150. Wireless network interface 145 can allow for communication with various wireless networks and/or wireless devices using one or more communication protocols. For example, wireless network interface 145 can allow for communication using a wireless local area network (WLAN), such as a WiFi-based network. In some embodiments, wireless network interface 145 allows for communication directly with other devices via Bluetooth, Bluetooth Low Energy (BLE), or some other device-to-device communication protocol. For example, wireless network interface 145 may allow for communication between BP measurement device 105 and remote sensing device 160.

Additionally or alternatively, other forms of wireless communication may be possible. For example, BP measurement device 105 may use a low-power wireless mesh network radio and protocol (e.g., Thread) to communicate with various smart home devices. In some embodiments, a wired network interface, such as an Ethernet connection, may be used for communication with a network. Further, the evolution of wireless communication to fifth generation (5G) and sixth generation (6G) standards and technologies provides greater throughput with lower latency which enhances mobile broadband services. 5G and 6G technologies also provide new classes of services, over control and data channels, for vehicular networking (V2X), fixed wireless broadband, and the Internet of Things (IoT). Such standards and technologies may be used for communication by BP measurement device 105.

A low-power wireless mesh network radio and protocol may be used for communicating with power limited devices. A power-limited device may be an exclusively battery powered device. Such devices may rely exclusively on one or more batteries for power and therefore, the amount of power used for communications may be kept low in order to decrease the frequency at which the one or more batteries need to be replaced. In some embodiments, a power-limited device may have the ability to communicate via a relatively high-power network (e.g., WiFi) and the low-power mesh network. The power-limited device may infrequently use the relatively high-power network to conserve power. Examples of such power-limited devices include environmental sensors (e.g., temperature sensors, carbon monoxide sensors, smoke sensors, motion sensors, presence detectors) and other forms of remote sensors.

Display 140 can allow processing system 110 to present information for viewing by one or more users. Speaker 150 can allow for sound, such as synthesized speech, to be output. For instance, responses to spoken commands received via microphone 134 may be output via speaker 150 and/or display 140. The spoken commands may be analyzed locally by BP measurement device 105 or may be transmitted via wireless network interface 145 to cloud-based server system 190 for analysis. A response, based on the analysis of the spoken command, can be sent back to BP measurement device 105 via wireless network interface 145 for output via speaker 150 and/or display 140. Additionally, or alternatively, the speaker 150 and microphone 134 may be collectively configured for active acoustic sensing, including ultrasonic acoustic sensing.

Notably, some embodiments of BP measurement device 105 do not have any still camera or video camera. By not incorporating an on-board camera, users nearby may be reassured about their privacy. For example, BP measurement device 105 can typically be installed in a user's bedroom. For many reasons, a user would not want a camera located in such a private space or aimed toward the user while the user is sleeping or performing some other private activity. In other embodiments, BP measurement device 105 may have a camera, but the camera's lens may be obscured by a mechanical lens shutter. In order to use the camera, the user may be required to physically open the shutter to allow the camera to have a view of the environment of BP measurement device 105. The user can be assured of privacy from the camera when the shutter is closed.

Remote sensing device 160 may generally be a smartwatch, fitness device, or other smart health device able to measure and/or monitor one or more vital signs from a user such as a user's body temperature, pulse, respiration, blood pressure, and the like, as well as other types of health data. Remote sensing device 160 may measure vital signs by sensing and/or collecting lower order data such as a person's oxygen saturation over time, electrical activity of the heart or brain, and the like. To sense and/or collect the data, remote sensing device 160 may be worn on a user's extremity as a watch or ring for extended periods of time. Additionally, or alternatively, remote sensing device 160 may be fitted to a user for a limited amount of time while vital sign measurements are being taken or performed.

Remote sensing device 160 can include: remote processing system 165; electronic display 162; wireless interface 164; one or more speakers 166; one or more microphones 168; and sensor suite 170. Remote processing system 165 may include one or more processors, which can include special-purpose or general-purpose processors that execute instructions stored using one or more non-transitory processor readable mediums. The instructions may configure the one or more processors of remote processing system 165 to perform various functions. For example, remote processing system 165 may be configured to control the operation of sensor suite 170 to collect sensor measurements from a user of remote sensing device 160. Remote processing system 165 may be further configured to analyze the sensor measurements to provide vital sign data, as discussed further below. Additionally, or alternatively, remote processing system 165 may be configured to receive the vital sign data directly from sensor suite 170 for subsequent processing, transmission, and/or output to a user.

Remote sensing device 160 may include one or more sensors and/or sensor subsystems configured to collect measurements associated with a user's health and/or vital signs. For example, remote sensing device 160 may include all, one, or some combination of the sensors provided as part of sensor suite 170. Sensor suite 170 can include: optical subsystem 172; inertial measurement unit (IMU) 174; temperature sensor 176; and electrical sensor 178. IMU 174 may allow remote sensing device 160 and/or remote processing system 165 to monitor movement and orientation remote sensing device 160. Monitoring the movement and orientation of remote sensing device 160 may allow remote processing system 165 to perform various functions, such as step counting, activity recognition, gesture control, and the like. Additionally, or alternatively, IMU 174 may allow remote processing system 165 to determine a relative position and orientation of remote sensing device 160 with respect to one or more external devices, such as BP measurement device 105.

IMU 174 may include multiple sensors, such as an accelerometer, a gyroscope, and a magnetometer. The accelerometer can measure linear acceleration, such as the movement of remote sensing device 160 up and down, left and right, or forward and backward. Additionally, or alternatively, the accelerometer can measure the direction acceleration due to earth's gravity. The gyroscope can measure angular velocity of remote sensing device 160, such as the rotation around a specific axis. The magnetometer can measure magnetic field strength and can be used to determine the orientation of remote sensing device 160 relative to the Earth's magnetic field.

Temperature sensor 176 may allow remote sensing device 160 and/or remote processing system 165 to measure the ambient temperature of the environment around remote sensing device 160 and/or the temperature of a user's skin adjacent to remote sensing device 160. Temperature sensor 176 may include one or more electrical sensors such as a thermistor, a thermocouple, a resistance temperature detector, or other similar components configured to detect changes in temperature and convert the changes in temperature to electrical signals. Additionally, or alternatively, temperature sensor 176 may include one or more optical sensors and/or use data from optical subsystem 172, to measure a user's temperature. For example, temperature sensor 176 may use one or more infrared emitters and receivers to measure the amount of infrared radiation emitted by a user's skin.

Electrical sensor 178 may allow remote sensing device 160 and/or remote processing system 165 to measure electrical signals and/or characteristics related to a user's body or environment, such as the electrical activity of a user's heart. Electrical sensor 178 may be, or otherwise include an electrocardiogram (ECG) sensor. Electrical sensor 178 may use two or more electrodes in contact with a user's skin to detect electrical signals generated by the user's heart as it beats. Voltage differences between the two or more electrodes may be measured to produce a waveform that represents the electrical activity of the user's heart. Subsequently, the waveform may be analyzed to determine the user's heart rate, heart rhythm, and other cardiac parameters. Additionally, or alternatively, the two or more electrodes may be used to detect moisture (e.g., perspiration) on a user's skin by measuring differences in electrical conductance of the user's skin.

Optical subsystem 172 may allow remote sensing device 160 to optically measure a person's pulse, respiration, and/or oxygen levels by detecting blood volume changes in the person's microvasculature. Optical subsystem 172 may be, or include, a photoplethysmography (PPG) sensor. Generally, as blood flows through the microvasculature of tissue, it causes small fluctuations in the blood volume of the tissue. As the blood volume of the tissue changes, the amount of light absorbed by the tissue changes. The amount of light absorbed by the tissue may also change as the oxygenation of the blood changes. Accordingly, by detecting the amount of light reflected by the tissue, and thereby absorbed by the tissue, optical subsystem 172 can be used to measure a user's pulse, respiration, and/or blood oxygen levels.

As further described below, optical subsystem 172 may include a light source or emitter, such as an LED, and a light receiver or photodetector, such as a photodiode or phototransistor. The light emitter may be configured to illuminate a user's skin, and the light receiver may be configured to measure changes in the intensity of the light as it passes through tissue of a user adjacent to the emitter and/or receiver. Remote sensing device 160 may be configured so that the light emitter and receiver are in contact with a user's skin during use. For example, in the case of wrist-worn device, such as a smart watch, the light emitter and receiver may be on an exterior surface of a housing of remote sensing device 160 on the interior of a loop formed by remote sensing device 160 and a wrist strap. When worn by a user, the light emitter and receiver may be held in contact with the skin of the user's wrist by pressure applied from the wrist strap.

While described herein as a wrist-worn device, other types of devices may be used in conjunction with optical subsystem 172, such as a ring, a finger clip, an arm band, and the like. Additionally, or alternatively, remote sensing device 160 and one or more components of optical subsystem 172 may be physically separate from each other. For example, remote sensing device 160 may be a smartphone, tablet, or laptop computer configured to receive wired and/or wireless communication from optical subsystem 172. Additionally, or alternatively, remote sensing device 160 may be configured to operate remote light emitter and receiver components of optical subsystem 172 via a wired and/or wireless connection to measure light reflected/absorbed by a user's tissue based on the intensity of light detected by the receiver.

Optical subsystem 172 may further include optical processing circuitry configured to analyze the changes in reflected light and determine one or more pulse wave measurements, such as pulse rate, pulse waveform, pulse pressure wave amplitude, and other vital sign data related to a person's cardiovascular health. Additionally, or alternatively, remote processing system 165 may be configured to receive the reflected light signals from optical subsystem 172 to derive the vital sign data related to the person's cardiovascular health. Subsequently, remote processing system 165 may transmit the reflected light signals and/or the vital sign data to an external device, such as BP measurement device 105 for additional processing. For example, BP measurement device 105 may perform various fusion analyses on the reflected light signals, pulse waveform, pulse rate, and/or other vital sign data from remote sensing device 160 along with other vital sign data to determine other health related metrics, such as a user's blood pressure.

In some embodiments, one or more additional sensors provided as part of sensor suite 170 may be used to compensate for changes in environmental conditions that may affect the accuracy of the pulse wave measurements. For example, a user's body temperature determined using temperature sensor 176 and/or perspiration determined using electrical sensor 178 may be used to improve the accuracy of the pulse wave measurements. Additionally, or alternatively, ambient temperature and/or humidity measurements collected by BP measurement device 105 may further supplement the accuracy of the pulse wave measurements.

Remote sensing device 160 may include interfaces such as electronic display 162, wireless interface 164, one or more speakers 166, and one or more microphones 168. Wireless interface 164 can allow for communication with wireless networks and/or wireless devices using the same or similar communication protocols described above in relation to wireless network interface 145. For example, wireless interface 164 can allow remote sensing device 160 to communicate with other devices, computer systems, and the like via WiFi, 5G, 6G, a wireless mesh network protocol, and the like. As another example, wireless interface 164 may allow for direct communication with other electronic devices, such as BP measurement device 105, via Bluetooth, Bluetooth Low Energy (BLE), or some other device-to-device communication protocol.

Remote sensing device 160 may be configured to communicate with other devices, such as BP measurement device 105, via wireless interface 164 to perform various health related measurements and procedures. For example, remote sensing device 160 may receive a signal, instructions, or a request from BP measurement device 105 to begin collecting and transmitting vital sign data related to a user's cardiovascular health, such as the user's pulse waveform as measured at the location of the remote sensing device 160 on the user's body. Additionally, or alternatively, remote sensing device 160 may transmit instructions or a request to BP measurement device 105 indicating that a user would like to perform a blood pressure measurement. In response, remote sensing device 160 and/or BP measurement device 160 may perform an initial synchronization between clocks of each respective device, as described further below. Once synchronized, remote sensing device 160 may begin collecting and transmitting the vital sign data to BP measurement device 105 while BP measurement device 105 begins collecting radar data from radar processing module 112. Using the synchronized data, BP measurement engine 114 may derive the user's BP measurements and subsequently transmit them to remote sensing device 160 for output to the user.

Electronic display 162, one or more speakers 166, and one or more microphones 168 can allow remote processing system 165 to provide various user interface functionalities. For example, electronic display 162 can allow remote processing system 165 to present various graphical user interfaces (GUIs). The GUIs may allow remote processing system 165 to present information to a user and/or receive inputs from a user. For example, the GUIs may display one or more selectable options, which when selected by a user, may configure remote processing system 165 and/or processing system 110 to begin collecting vital sign data from the user. The GUIs may further be used to display information to a user, such as instructions for performing an accurate collection of vital sign data. For example, remote processing system 165 may display instructions to a user regarding the appropriate position, posture, activity level, and the like of either the user or the remote sensing device 160 that may result in improved measurement readings.

One or more microphones 168 can allow remote processing system 165 to present various voice user interfaces (VUIs). Using speech recognition technology, remote processing system 165 may be configured to monitor for one or more spoken commands detectable in audio captured by one or more microphones. For example, the VUIs may allow a user to speak a command corresponding to instructions to begin collecting vital sign data. In response, one or more speakers 166 may output audio confirming the user's instructions and/or providing further instructions for performing the vital sign data collection. As described above in reference to BP measurement device 105, remote sensing device 160 may perform speech recognition locally and/or transmit the detected audio to a remote processing system, such as BP measurement device 105 and/or cloud-based server system 190, for analysis and response generation.

As described above, wireless network interface 145 and/or wireless interface 164 can allow for wireless communication with network 180. Network 180 can include one or more public and/or private networks. Network 180 can include a local wired or wireless network that is private, such as a home wireless local area network. Network 180 may also include a public network, such as the Internet. Network 180 can allow for communication between BP measurement device 105 and remote sensing device 160. Additionally, or alternatively, network 180 can allow BP measurement device 105 and/or remote sensing device 160 to communicate with remotely located cloud-based server system 190.

Cloud-based server system 190 can provide BP measurement device 105 and/or remote sensing device 160 with various services. Regarding BP data, cloud-based server system 190 can include processing and storage services for BP-related data. While the embodiment of FIG. 1 involves processing system 110 performing BP measurement, such functions may be performed by cloud-based server system 190. Further, in addition to, or instead of, using BP data storage 118 to store BP data, BP-related data may be stored by cloud-based server system 190, such as mapped to a common user account to which BP measurement device 105 is linked. If multiple users are associated with BP measurement device 105, the BP data may be stored and mapped to individual user accounts, such as user accounts associated with remote sensing device 160.

Regardless of whether a single user or multiple users use BP measurement device 105 to measure BP, each user may be required to provide their informed consent. Such informed consent may involve each user consenting to an end user agreement that involves data being used in compliance with HIPAA and/or other generally accepted security and privacy standards for health information. Periodically, users may be required to renew their consent to collection of BP data, such as annually. In some embodiments, each end user may receive a periodic notification, such as via a mobile device (e.g., smartphone) that reminds each user that their BP data is being collected and analyzed and offers each user the option to disable such data collection.

Cloud-based server system 190 may additionally or alternatively provide other cloud-based services. For instance, BP measurement device 105 may additionally function as a home assistant device. A home assistant device may respond to vocal queries from a user. In response to detecting a vocal trigger phrase being spoken, BP measurement device 105 may record audio via microphone 134. A stream of the audio may be transmitted to cloud-based server system 190 for analysis. Cloud-based server system 190 may perform a speech recognition process, use a natural language processing engine to understand the query from the user, and provide a response to be output by BP measurement device 105 as synthesized speech, an output to be presented on display 140, and/or a command to be executed by BP measurement device 105 (e.g., raise the volume of BP measurement device 105) or sent to some other device, such as remote sensing device 160. Further, queries or commands may be submitted to cloud-based server system 190 via display 140, which may be a touchscreen. For instance, BP measurement device 105 may be used to control various smart home devices or home automation devices. Such commands may be sent directly by BP measurement device 105 to the device to be controlled or may be sent via cloud-based server system 190.

FIG. 2 illustrates a block diagram of an embodiment of a sensor-fusion-based BP measurement system 200 (“system 200”). System 200 can include radar subsystem 205 (which can represent an embodiment of radar subsystem 120); radar processing module 210 (which can represent an embodiment of radar processing module 112); optical subsystem 220 (which can represent an embodiment of optical subsystem 172); remote sensor module 225 (which can represent an embodiment of remote sensor module 113); and BP measurement engine 230 (which can represent an embodiment of BP measurement engine 114).

Radar subsystem 205 may include RF emitter 206, RF receiver 207, and radar processing circuit 208. RF emitter 206 can emit radio waves, such as in the form of continuous-wave (CW) radar. RF emitter 206 may use frequency-modulated continuous-wave (FMCW) radar. The FMCW radar may operate in a burst mode or continuous sparse-sampling mode. In burst mode, a frame or burst of multiple chirps, with the chirps spaced by a relatively short period of time, may be output by RF emitter 206. Each frame may be followed by a relatively long amount of time until a subsequent frame. In a continuous sparse-sampling mode, frames or bursts of chirps are not output, rather chirps are output periodically. The spacing of chirps in the continuous sparse sampling mode may be greater in duration than the spacing between chirps within a frame of the burst mode. In some embodiments, radar subsystem 205 may operate in a burst mode, but output raw chirp waterfall data for each burst may be combined (e.g., averaged) together to create simulated continuous sparse-sampled chirp waterfall data. In some embodiments, raw waterfall data gathered in burst mode may be preferable for gesture detection while raw waterfall data gathered in a continuously sparse sampling mode may be preferable for certain functions, such as BP measurement, sleep tracking, vital sign detection, and, generally, health monitoring. Gesture detection may be performed by other hardware or software components that use the output of radar subsystem 205 that are not illustrated.

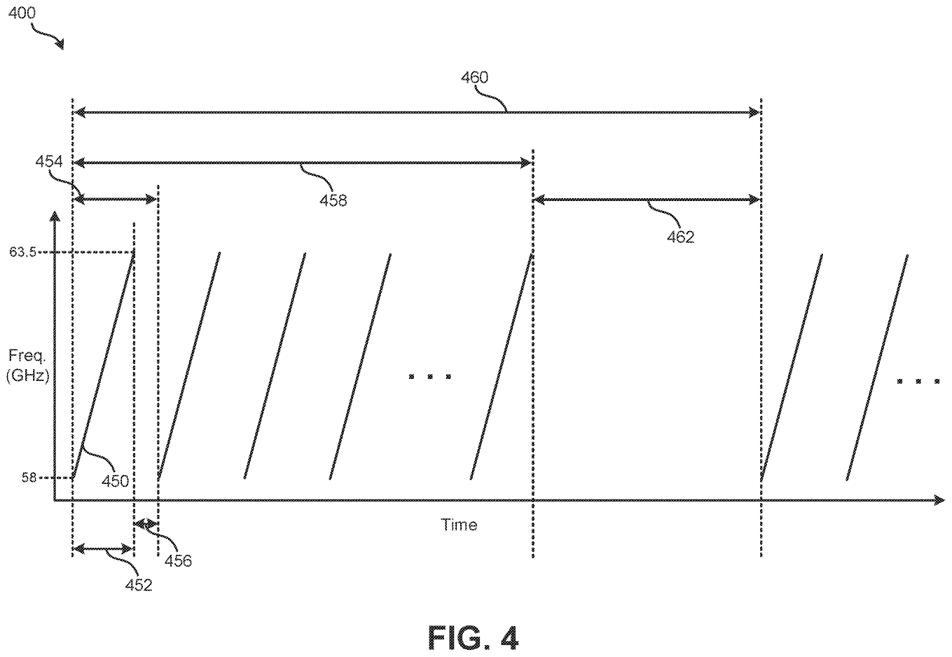

RF emitter 206 may include one or more antennas and may transmit at or about 60 GHz. The frequency of radio waves transmitted may repeatedly sweep from a low to high frequency (or the reverse). The power level used for transmission may be very low such that radar subsystem 205 has an effective range of several meters or an even shorter distance. Further detail regarding the radio waves generated and emitted by radar subsystem 205 are provided in relation to FIG. 4.

RF receiver 207 includes one or more antennas, distinct from the transmit antenna(s), and may receive radio wave reflections off of nearby objects of radio waves emitted by RF emitter 206. The reflected radio waves may be interpreted by radar processing circuit 208 by mixing the radio waves being transmitted with the reflected received radio waves, thereby producing a mixed signal that can be analyzed for distance. Based on this mixed signal, radar processing circuit 208 may output raw waveform data, which can also be referred to as the waterfall data for analysis by a separate processing entity. Radar subsystem 205 may be implemented as a single integrated circuit (IC) or radar processing circuit 208 may be a separate component from RF emitter 206 and RF receiver 207. In some embodiments, radar subsystem 205 is integrated as part of device 105 such that RF emitter 206 and RF receiver 207 are pointing in a same direction as display 140. In other embodiments, an external device that includes radar subsystem 205 may be connected with device 105 via wired or wireless communication. For example, radar subsystem 205 may be an add-on device to a home assistant device.

Raw waveform data may be passed from radar subsystem 205 to radar processing module 210. The raw waveform data passed to radar processing module 210 may include waveform data indicative of continuous sparse reflected chirps due to radar subsystem 205 operating in a continuous sparse sampling mode or due to radar subsystem 205 operating in a burst mode and a conversion process to simulate raw waveform data produced by radar subsystem 205 operating in a continuous sparse sampling mode being performed. Processing may be performed to convert burst sampled waveform data to continuous sparse samples using an averaging process, such as each reflected group of burst radio waves being represented by a single averaged sample. Radar processing module 210 may include one or more processors. Radar processing module 210 may include one or more special-purpose or general-purpose processors. Special-purpose processors may include processors that are specifically designed to perform the functions detailed herein.

Such special-purpose processors may be ASICs or FPGAs which are general-purpose components that are physically and electrically configured to perform the functions detailed herein. General-purpose processors may execute special-purpose software that is stored using one or more non-transitory processor-readable mediums, such as random access memory (RAM), flash memory, a hard disk drive (HDD), or a solid state drive (SSD). Radar processing module 210 may include: movement filter 211; frequency emphasizer 212; range-vitals transform engine 213; range gating filter 214; spectral summation engine 215; and radar timing engine 216. Each of the components of radar processing module 210 may be implemented using software, firmware, or as specialized hardware.

The raw waveform data output by radar subsystem 205 may be received by radar processing module 210 and first processed using movement filter 211. In some embodiments, it is important that movement filter 211 is the initial component used to perform filtering. That is, the processing performed by radar processing module 210 is not commutative in some embodiments. Typically, vital sign determination (which includes BP measurement) may occur when a monitored user is in a low movement environment. In such an environment, there may typically be little movement. For what movement is present, the movement may be attributed to the user's vital signs, including movement due to breathing and movement due to the monitored user's heartbeat. In such an environment, a large portion of emitted radio waves from RF emitter 206 may be reflected by static objects in the vicinity of the monitored user, such as furniture, bedding, walls, etc. Therefore, a large portion of the raw waveform data received from radar subsystem 205 may be unrelated to user movements and the user's vital measurements.

Movement filter 211 may include a waveform buffer that buffers “chirps” or slices of received raw waveform data. For instance, sampling may occur at a rate of 10 Hz. In other embodiments, sampling may be slower or faster. Movement filter 211 may buffer twenty seconds of received raw waveform chirps in certain embodiments. In other embodiments, a shorter or longer duration of buffered raw waveform data is buffered. This buffered raw waveform data can be filtered to remove raw waveform data indicative of stationary objects. That is, for objects that are moving, such as a monitored user's chest, the user's heartbeat and breathing rate will affect the distance and velocity measurements made by radar subsystem 205 and output to movement filter 211. This movement of the user will result in “jitter” in the received raw waveform data over the buffered time period. More specifically, jitter refers to the phase shifts caused by moving objects reflecting emitted radio waves. Rather than using the reflected FMCW radio waves to determine a velocity of the moving objects, the phase shift induced by the motion in the reflected radio waves can be used to measure vital statistics, including heartrate and breathing rate, as detailed herein.

For stationary objects, such as furniture, a zero phase shift (i.e., no jitter) will be present in the raw waveform data over the buffered time period. Movement filter 211 can subtract out such raw waveform data corresponding to stationary objects such that motion-indicative raw waveform data is passed to frequency emphasizer 212 for further analysis. Raw waveform data corresponding to stationary objects may be discarded or otherwise ignored for the remainder of processing by radar processing module 210.

In some embodiments, an infinite impulse response (IIR) filter is incorporated as part of movement filter 211. Specifically, a single-pole IIR filter may be implemented to filter out raw waveform data that is not indicative of movement. Therefore, the single-pole IIR filter may be implemented as a high-pass, low-block filter that prevents raw waveform data indicative of movement below a particular frequency from passing through to frequency emphasizer 212. The cut-off frequency may be set based on known limits to human vital signs. For example, a breathing rate may be expected to be between 10 and 60 breaths per minute. Movement data indicative of a lower frequency than 10 breaths per minute may be excluded by the filter. In some embodiments, a band-pass filter may be implemented to exclude raw waveform data indicative of movement at high frequencies that are impossible or improbable for human vital signs. For instance, a heartrate, which can be expected to be above a breathing rate, may be unlikely to be above 150 beats per minute for a person in a resting or near-resting state. Raw waveform data indicative of a higher frequency may be filtered out by the band pass filter.

The vital signs of the monitored user being measured are periodic impulse events: a user's heartrate may vary over time, but it can be expected that the user's heart will continue to beat periodically. This beating is not a sinusoidal function, but rather may be understood as an impulse event, more analogous to a square wave having a relatively low duty cycle that induces motion in the user's body. Frequency components due to this spectral leakage should be deemphasized.

Frequency emphasizer 212 may work in conjunction with range-vitals transform engine 213 to determine the frequency components of the raw waveform data attributable to vital statistics, such as a pulse of a user. Frequency emphasizer 212 may use frequency windowing, such as a 2D Hamming window (other forms of windowing are possible, such as a Hann window), to emphasize important frequency components of the raw waveform data and to deemphasize or remove waveform data that is attributable to spectral leakage outside of the defined frequency window. Such frequency windowing may decrease the magnitude of raw waveform data that is likely due to processing artifacts. The use of frequency windowing can help reduce the effects of data-dependent processing artifacts while preserving data relevant for being able to separately determine heartrate and breathing rate.

For a stationary tabletop or bedside FMCW radar-based monitoring device, which may be positioned within 1 to 2 meters of the one or more users being monitored to detect heartrate, a 2D Hamming window that emphasizes frequencies in the range of 30 to 150 bpm (0.5 to 2.5 Hz) for heartbeat provides for sufficiently good signals to make reliable measurements without requiring advance knowledge of the subject's age or medical history.

Since heartrate and breathing rate are periodic impulse events, the frequency domain heartrate, and breathing rate may be represented by different fundamental frequencies, but each may have many harmonic components at higher frequencies. One of the primary purposes of frequency emphasizer 212 may be to prevent the frequency ripples of harmonics of the monitored user's heartrate from affecting any other vital statistic, such as breathing rate, being measured. While frequency emphasizer 212 may use a 2D Hamming window, it should be understood that other windowing functions or isolating functions can be used to help isolate frequency ripples of the monitored user's breathing rate from the frequency ripples of the monitored user's heartrate.

Range-vitals transform engine 213 analyzes the received motion-filtered waveform data to identify and quantify the magnitude of movement at specific frequencies. More particularly, range-vitals transform engine 213 analyzes phase jitter over time to detect relatively small movements due to a user's vital signs that have a relatively low frequency, such as breathing rate and heart rate. The analysis of range-vitals transform engine 213 may assume that the frequency components of the motion waveform data are sinusoidal. Further, the transform used by range-vitals transform engine 213 can also identify the distance at which the frequency is observed. Frequency, magnitude, and distance can all be determined at least in part because radar subsystem 205 uses an FMCW radar system.

Range-vitals transform engine 213 can perform a series of Fourier transform (FT) to determine the frequency components of the received raw waveform data output by frequency emphasizer 212. Specifically, a series of fast Fourier transform (FFT) may be performed by range-vitals transform engine 213 to determine the specific frequencies and magnitudes of waveform data at such frequencies.

Waveform data obtained over a period of time can be expressed in multiple dimensions. A first dimension (e.g., along the y-axis) can relate to multiple samples of waveform data from a particular chirp and a second dimension (e.g., along the x-axis) relates to a particular sample index of waveform data gathered across multiple chirps. A third dimension of data (e.g., along the z-axis) is present indicative of the intensity of the waveform data.

Multiple FFTs may be performed based on the first and second dimension of the waveform data. FFTs may be performed along each of the first and second dimensions: an FFT may be performed for each chirp and an FFT may be performed for each particular sample index across multiple chirps that occurred during the period of time. An FFT performed on waveform data for a particular reflected chirp can indicate one or more frequencies, which, in FMCW radar, are indicative of the distances at which objects are present that reflected emitted radio waves. An FFT performed for a particular sample index across multiple chirps can measure the frequency of phase jitter across the multiple chirps. Therefore, the FFT of the first dimension can provide the distance at which a vital statistic is present and the FFT of the second dimension can provide a frequency of the vital statistic. The output of the FFTs performed across the two dimensions is indicative of: 1) the frequencies of vital statistics; 2) the ranges at which the vital statistics were measured; and 3) the magnitudes of the measured frequencies. In addition to values due to vital statistics being present in the data, noise may be present that is filtered, such as using spectral summation engine 215. The noise may be partially due to heartrate and breathing not being perfect sinusoidal waves.

To be clear, the transform performed by range-vitals transform engine 213 differs from a range-Doppler transform. Rather than analyzing changes in velocity (as in a range-Doppler transform), periodic changes in phase shift over time are analyzed as part of the range-vitals transform. The range-vitals transform is tuned to identify small movements (e.g., breathing, expansion of blood vessels and valves) occurring over a relatively long period of time by tracking changes in phase, referred to as phase jitter.

Range gating filter 214 is used to monitor a defined range of interest and exclude waveform data due to movement beyond the defined range of interest. For arrangements detailed herein, the defined range of interest may be 0 to 1 meter. In some embodiments, this defined range of interest may be different or possibly set by a user (e.g., via a training or setup process) or by a service provider. In some embodiments, a goal of this arrangement may be to monitor the one person closest to the device (and exclude or segregate data for any other person farther away, such as a person sleeping next to the person being monitored). Therefore, range-vitals transform engine 213 and range gating filter 214 serve to segregate, exclude, or remove movement data attributed to objects outside of the defined range of interest and sum the energy of movement data attributed to objects within the defined range of interest. The output of range gating filter 214 may include data that has a determined range within the permissible range of range gating filter 214. The data may further have a frequency dimension and a magnitude. Therefore, the data may possess three dimensions.

As part of range-vitals transform engine 213 and/or range gating filter 214, data gathered at particular distances may be binned together. For example, some number of range bins may be created, such as 256. Motion that is detected within a particular distance range corresponding to a bin may be grouped together for analysis. As will be detailed herein, a first distance bin may be identified at which a user's pulse is detected at or near the user's aortic valve; a second distance bin may be identified at which a user's pulse is detected at a user's extremity (e.g., hands, feet).

Spectral summation engine 215 may receive the output from range gating filter 214. Spectral summation engine 215 may function to transfer the measured energy of harmonic frequencies of the user's heartrate and sum the harmonic frequency energy onto the fundamental frequency's energy for each distance bin. This function can be referred to as a harmonic sum spectrum (HSS). As previously noted, vital statistics such as heartrate and breathing rate are not sinusoidal; therefore, in the frequency domain, harmonics will be present at frequencies higher than the fundamental frequency of the user's breathing rate and the fundamental frequency of the user's heartrate. One of the primary purposes of spectral summation engine 215 can be to prevent harmonics of the monitored user's breathing rate from affecting the frequency measurement of the monitored user's heartrate (and the reverse). The HSS may be performed at the second order by summing the original spectrum with a down-sampled instance (by a factor of two) of the spectrum. This process may also be applied at higher order harmonics such that their respective spectra are added to the spectrum at the fundamental frequency. The HSS may be applied for some or all distance bins in which a pulse (and/or breathing) is detected.

Radar timing engine 216 may work in combination with clock synchronization engine 226 and/or optical timing engine 227, as further described below, to synchronize the output of radar processing module 210 with other inputs to BP measurement engine 230. From the perspective of a clock associated with an electronic device, there may be a delay between the time at which a real-world event occurred, such as a heartbeat, and the time at which data from a sensor observing the event is finally processed and assigned a timestamp. This delay may be as a result of physical constraints associated with electronic sensors, processing power capabilities, algorithmic complexities, and the like. To account for this delay, radar timing engine 216 may adjust timestamps associated with the output of radar processing module 210. For example, radar timing engine 216 may apply a static adjustment amount to each timestamp based on predefined specifications associated with the particular device and/or components therein, such as a model of a processor or sensor, a version of software, and the like. Additionally, or alternatively, radar timing engine 216 may monitor one or more operating characteristics of the electronic device, such as current processor loads, and/or characteristics of the underlying data, such as a distance between the electronic device and a user, to dynamically calculate an adjustment amount.

Optical subsystem 220 may include light emitter 221, light receiver 222, and optical processing circuit 223. As described above in relation to optical subsystem 172, light emitter 221 may include one or more sources of light, such as an LED, a laser diode, an organic LED (OLED, and the like configured to illuminate a user's skin. Light emitter 221 may be configured to emit light at one or more specific wavelengths, such as red or infrared, that may be absorbed and/or reflected differently by oxygenated and deoxygenated blood. As further described above, light receiver 222 may include one or more photodetectors, such as a photodiode and/or phototransistor, configured to convert incoming light at the wavelengths emitted by light emitter 221 into an electrical signal. Optical processing circuit 223 may process the electrical signal from light receiver 222 to detect the amount of light from light emitter 221 reflected or absorbed by tissue of a user. Optical processing circuit 223 may represent the amount of light reflected or absorbed by the tissue of the user in a raw waveform. Additionally, or alternatively, optical processing circuit 223 may further analyze the amount of reflected or absorbed light over time to derive a pulse waveform.

Optical subsystem 220 may be implemented as a single integrated circuit (IC) or optical processing circuit 223 may be a separate component from light emitter 221 and light receiver 222. In some embodiments, optical subsystem 205 is integrated as part of remote sensing device 160 such that light emitter 221 and light receiver 222 are pointing in an opposite direction as display 162. In other embodiments, an external device that includes one or more components of optical subsystem 220 may be connected with remote sensing device 160 and/or BP measurement device 105 via wired or wireless communication.

Pulse waveform data may be passed from optical subsystem 220 to remote sensor module 225. The pulse waveform data passed to remote sensor module 225 may include waveform data indicative of light intensity (e.g., light absorption) measured at an extremity of a user. Additionally, or alternatively, the pulse waveform data may include a processed pulse waveform measured at the extremity of the user. As described above in relation to radar processing module 210, remote sensor module 225 may include one or more general-purpose or special-purpose processors. Additionally, or alternatively, remote sensor module 225 and radar processing module 210 may share a processing system. For example, remote sensor module 226 and radar processing module 210 may be separate firmware or software components executing on a same device, such as BP measurement device 105. Additionally, or alternatively, one or more components or functions of remote sensor module 226 may be executed on, or facilitated by, a same device as optical subsystem 220, such as remote sensing device 160.

Remote sensor module 225 may include: clock synchronization engine 226 and optical timing engine 227. As described above in reference to radar timing engine 216, optical timing engine 227 may adjust timestamps associated with the output of optical subsystem 220 to account for delays between the time at which a pulse wave is detectable by optical subsystem 220 and the time at which the pulse wave is actually recorded in data associated with a timestamp. For example, optical timing engine 227 may adjust timestamps based on a model, version, or manufacturer of the electronic device and/or optical subsystem 220.

In addition to processing delays between the time at which a real-world event occurs and the time at which data representing the real-world event is recorded, the accuracy of measurements derived from data collected by distributed systems may be further degraded due to differences in the chronological reference frame for each system. As described herein, the chronological reference frame for each system is assumed to be with respect to a clock of each system. Such differences may arise as a result of each clock being calibrated to a different reference source, such as Global Positioning System (GPS) time, Coordinated Universal Time (UTC), or the like, inaccurate initial time calibration (e.g., with respect to the time reference source), and/or subsequent clock drift.

Clock synchronization engine 226 may account for the differences in the chronological reference frame used by radar subsystem 205, radar processing module 210, and/or optical subsystem 220 by synchronizing the clocks of each reference frame to a common time reference. For example, in the case of two electronic devices (e.g., BP measurement device 105 and remote sensing device 160), clock synchronization engine 226 may synchronize a first clock of remote sensing device 160 to a second clock of BP measurement device 105 or vice versa. Alternatively, clock synchronization engine 226 may synchronize clocks to a standard time reference source, such as UTC or GPS time. Based on the common time reference, clock synchronization engine 226 may synchronize the clocks using one or more techniques, such as Network Time Protocol (NTP), Precision Time Protocol (PTP), and the like.

The time adjusted outputs of radar processing module 210 and remote sensor module 225 can be passed to BP measurement engine 230. BP measurement engine 230, which may be implemented using the same or a different processing system as radar processing module 210 and/or remote sensor module 225, can include: radar analysis engine 231, optical analysis engine 232, PTT determination engine 233; and BP determination engine 234.

Radar analysis engine 231 can serve to detect a pulse waveform due to the user's heartbeat at the user's chest using the output from radar processing module 210. More specifically, radar analysis engine 231 may detect the pulse waveform at or near the aortic value of the user. Radar analysis engine 231 may analyze the range binned data received from spectral summation engine 215. Since a heartbeat causes a large amount of movement at and around an aortic value, a distance bin having a greatest amount of movement attributed to the pulse waveform can be the distance bin selected and analyzed by radar analysis engine 231. Radar analysis engine 231 can be a trained machine learning model, such as a trained neural network, that can identify a heartbeat of the user (e.g., a pulse waveform) and can assign timestamps to the heartbeat based on the time adjusted and synchronized output from radar processing module 210.

In some embodiments, radar analysis engine 231 further detects a pulse waveform due to the user's heartbeat at an extremity of the user, such as the user's hand(s), or feet. For example, a user may be requested to set their hands or feet a defined distance from radar subsystem 205 such that a distance bin corresponding to the user's extremity may be known. Based on the position in which the user is requested to hold their hands or feet, the distance bin determined for the user's extremity may be the closest distance bin to radar subsystem 205 at which the pulse can be detected. Based on the data in the distance bin corresponding to the user's extremity, radar analysis engine 231 can identify and assign timestamps to the pulse waveform at the user's extremity.

Optical analysis engine 232 can serve to detect a pulse waveform due to the user's pulse at the location on the user's body adjacent to the light emitter 221 and/or light receiver 222. As described herein, this location is assumed to be an extremity of the user, such as the user's wrist, finger, hand, foot, or arm. Optical analysis engine 232 may analyze the time adjusted and synchronized output from remote sensor module 225 to identify and assign timestamps to the pulse waveform at the user's extremity. For example, optical analysis engine 232 may apply one or more signal processing algorithms to a light absorption/refection waveforms to derive a pulse waveform. Such algorithms may include filters, noise reduction techniques, peak detection algorithms, and the like. Additionally, or alternatively, optical analysis engine 232 may be configured to receive a pulse waveform provided by optical subsystem 220.

PTT determination engine 233 can determine at least the PTT by calculating a difference in time between the pulse waveform measured at the user's chest by radar analysis engine 231 and the pulse waveform (attributed to the same heartbeat) measured at the user's extremity by optical analysis engine 232 and/or radar analysis engine 231. Additionally, or alternatively, PTT determination engine 233 may initially calculate a phase difference between the pulse waveform measured by radar analysis engine 231 and the pulse waveform measured by optical analysis engine 232 from which the PTT can be determined. In some embodiments, additional data based on the pulse waveform measured by radar analysis engine 231 and/or optical analysis engine 232 can be determined. This data can include a derived pulse waveform amplitude (DPWA), which is indicative of the magnitude of the pulse as measured at the user's extremity and/or chest. In some embodiments, separate DPWA values are calculated for the extremity and chest. This data can additionally or alternatively include heartrate (HR) data by determining a number of pulse waveforms over a period of time. In some embodiments, separate HR values are calculated for the extremity and chest. Additionally, or alternatively, a respiration rate (RR) can be derived by identifying periodic increases and matching decreases in HR and/or the pulse waveform frequency. PTT, HR, DPWA, RR, or some combination thereof may be output to BP determination engine 234.

BP determination engine 234 may be an algorithm or look-up arrangement that uses PTT, HR, DPWA, and/or RR to calculate a BP, which can include systolic and diastolic blood pressures. In some embodiments, a trained machine learning model can receive these values as an input and can output systolic and diastolic blood pressures. In some embodiments, a calibration profile is applied to adjust the determined BP based on one or more measurements of the user's BP taken via another form of BP measurement device. The output of BP determination engine 234 may be output for presentation via display 140, display 162, speaker 150, one or more speakers 166, and/or stored by BP data storage 118 and/or cloud-based server system 190.

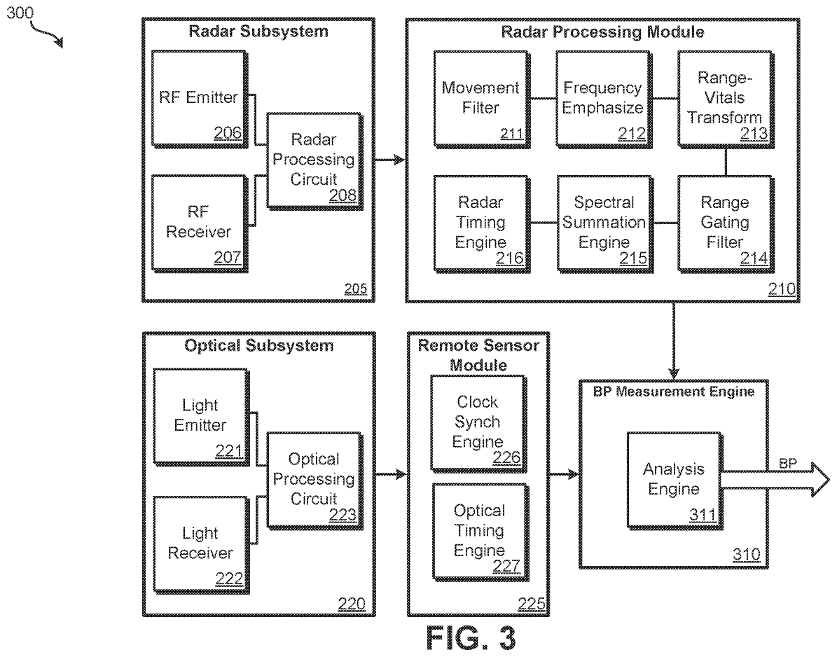

FIG. 3 illustrates a block diagram of an embodiment of a sensor-fusion-based BP measurement system 300 (“system 300”). System 300 can include radar subsystem 205 (which can represent an embodiment of radar subsystem 120); radar processing module 210 (which can represent an embodiment of radar processing module 112); optical subsystem 220 (which can represent an embodiment of optical subsystem 172); remote sensor module 225 (which can represent an embodiment of remote sensor module 113); and BP measurement engine 310 (which can represent an embodiment of BP measurement engine 114). System 300 may function similarly to system 200 with the exception of BP measurement engine 310. BP measurement engine 310 may include only analysis engine 311. Analysis engine 311 can be a trained machine learning model, such as a neural network, that receives the time adjusted and synchronized outputs of radar processing module 210 and remote sensor module 225 as its input. Analysis engine 311 may be trained to output BP measurements, such as systolic and diastolic values, by directly analyzing the outputs of radar processing module 210 and remote sensor module 225.

To train analysis engine 311, a large number of measurements of users may be made using a distributed system similar to radar processing module 210 and remote sensor module 225. The output data may be mapped to a blood pressure of the users gathered using an accurate BP measurement device. This training data may then be used to construct a machine learning model that can determine blood pressure based on the output of radar processing module 210 and remote sensor module 225.

In some embodiments, a calibration profile is applied to adjust the determined BP based on one or more measurements of the user's BP taken via another form of BP measurement device. In system 300, a layer of analysis engine 311 can be created or altered to account for the user's calibration profile.

FIG. 4 illustrates an embodiment of chirp timing diagram 400 for frequency modulated continuous wave (FMCW) radar radio waves output by a radar subsystem. Chirp timing diagram 400 is not to scale. Radar subsystem 205 may generally output radar in the pattern of chirp timing diagram 400. Chirp 450 represents a continuous pulse of radio waves that sweeps up in frequency from a low frequency to a high frequency. In other embodiments, individual chirps may continuously sweep down from a high frequency to a low frequency, from a low frequency to a high frequency, and back to a low frequency, or from a high frequency to a low frequency and back to a high frequency. In some embodiments, the low frequency is 58 GHz and the high frequency is 63.5 GHz. (For such frequencies, the radio waves may be referred to as millimeter waves.) In some embodiments, the frequencies are between 57 and 64 GHz. The low frequency and the high frequency may be varied by embodiment. For instance, the low frequency and the high frequency may be between 45 GHz and 80 GHz. The frequencies may be selected at least in part to comply with governmental regulation. In some embodiments, each chirp includes a linear sweep from a low frequency to a high frequency (or the reverse). In other embodiments, an exponential or some other pattern may be used to sweep the frequency from low to high or high to low.

Chirp 450, which can be representative of all chirps in chirp timing diagram 400, may have chirp duration 452 of 128 μs. In other embodiments, chirp duration 452 may be longer or shorter, such as between 50 μs and 1 ms. In some embodiments, a period of time may elapse before a subsequent chirp is emitted. Inter-chirp pause 456 may be 405.33 μs. In other embodiments, inter-chirp pause 456 may be longer or shorter, such as between 10 μs and 1 ms. In the illustrated embodiment, chirp period 454, which includes chirp 450 and inter-chirp pause 456, may be 333.33 μs. This duration varies based on the selected chirp duration 452 and inter-chirp pause 456.

A number of chirps that are output, separated by inter-chirp pauses, may be referred to as frame 458 or frame 458. Frame 458 may include twenty chirps. In other embodiments, the number of chirps in frame 458 may be greater or fewer, such as between 1 and 100. The number of chirps present within frame 458 may be determined based upon a maximum amount of power that is desired to be output within a given period of time. The FCC or other regulatory agency may set a maximum amount of power that is permissible to be radiated into an environment. For example, a duty cycle requirement may be present that limits the duty cycle to less than 10% for any 33 ms time period. In one particular example in which there are twenty chirps per frame, each chirp can have a duration of 128 μs, and each frame can be 33.33 ms in duration. The corresponding duty cycle is (20 frames)*(0.128 ms)/(33.33 ms), which is about 8.8%. By limiting the number of chirps within frame 458 prior to an inter-frame pause, the total amount of power output may be limited. In some embodiments, the peak effective isotropically radiated power (EIRP) may be 13 dBm (20 mW) or less, such as 12.86 dBm (19.05 mW). In other embodiments, the peak EIRP is 15 dBm or less and the duty cycle is 15% or less. In some embodiments, the peak EIRP is 40 dBm or less. That is, at any given time, the amount of power radiated by the radar subsystem might never exceed such values. Further, the total power radiated over a period of time may be limited.

Frames may be transmitted at a frequency of 30 Hz (33.33 ms) as shown by time period 460. In other embodiments, the frequency may be higher or lower. The frame frequency may be dependent on the number of chirps within a frame and the duration of inter-frame pause 462. For instance, the frequency may be between 1 Hz and 50 Hz. In some embodiments, chirps may be transmitted continuously, such that the radar subsystem outputs a continuous stream of chirps interspersed with inter-chirp pauses. Tradeoffs can be made to save on the average power consumed by the device due to transmitting chirps and processing received reflections of chirps. Inter-frame pause 462 represents a period of time when no chirps are output. In some embodiments, inter-frame pause 462 is significantly longer than the duration of frame 458. For example, frame 458 may be 6.66 ms in duration (with chirp period 454 being 333.33 μs and 40 chirps per frame). If 33.33 ms occur between frames, inter-frame pause 462 may be 46.66 ms. In other embodiments, the duration of inter-frame pause 462 may be larger or smaller, such as between 15 ms and 40 ms.

In the illustrated embodiment of FIG. 4, a single frame 458 and the start of a subsequent frame are illustrated. It should be understood that each subsequent frame can be structured similarly to frame 458. Further, the transmission mode of the radar subsystem may be fixed. That is, regardless of whether a user is present or not, the time of day, or other factors, chirps may be transmitted according to chirp timing diagram 400. Therefore, in some embodiments, the radar subsystem always operates in a single transmission mode, regardless of the state of the environment or the activity attempting to be monitored. A continuous train of frames similar to frame 458 may be transmitted while BP measurement device 105 is powered on.

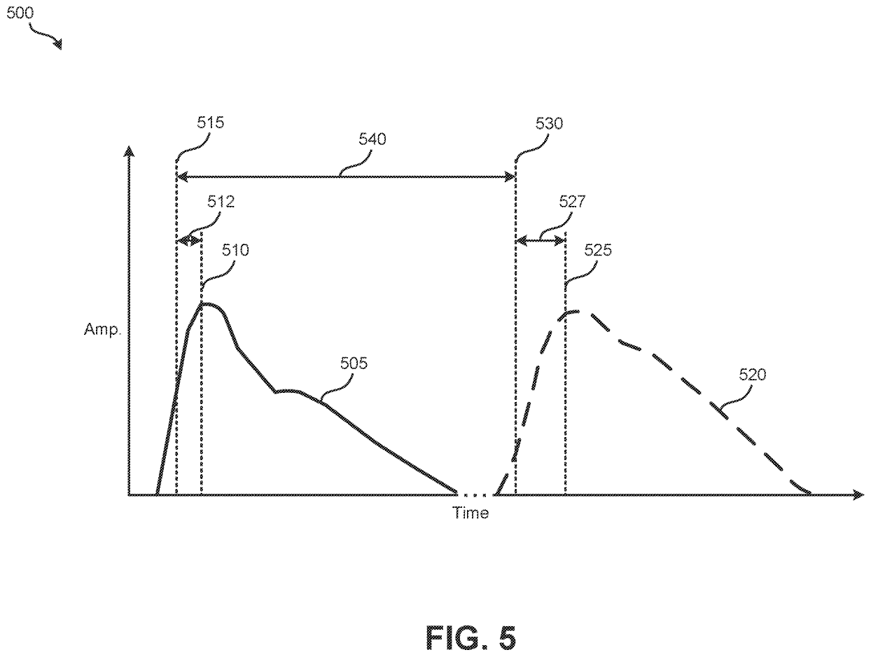

FIG. 5 illustrates a simplified graph 500 of a pulse wave measured by a distributed BP measurement system. As described above, components of a distributed BP measurement system, such as radar subsystem 205 and optical subsystem 220, may be used to detect pulse waveforms at different locations of a user's body. For example, radar analysis engine 231 can detect aortic pulse wave 505 at the user's chest using the output from radar processing module 210. As another example, optical analysis engine 232 may detect peripheral pulse wave 520 at an extremity of the user, such as the user's wrist or finger. For simplicity, aortic pulse wave 505 and peripheral pulse wave 520 are illustrated as singular pulse waves corresponding to a same singular heartbeat. However, it should be clear that each pulse wave may be part of a continuous waveform measured at the chest and extremity of the user respectively. Furthermore, aortic pulse wave 505 and peripheral pulse wave 520 are substantially separated from each other for illustration purpose so that they do not overlap with each other. However, in most instances, there would be considerable overlap in the time-domain (e.g., along the x-axis) between a pulse wave detected at the chest and the same pulse wave detected at an extremity.

As further illustrated in graph 500, aortic pulse wave 505 may be detected at first time 510 and peripheral pulse wave 525 may be detected at second time 525. First time 510 and second time 525 may each correspond to the time at which a peak or maxima of each respective pulse wave is detected by the system. While described in reference to a peak or maxima of each respective pulse wave, other points within a pulse wave may instead be used as reference points from which subsequent determinations and/or measurements may be made, such as the start of each pulse wave corresponding to the systolic upstroke, the dicrotic notch, and the like.

As further described above in reference to radar timing engine 216 and/or optical timing engine 227, there may be a delay between the time at which a pulse wave occurred or was otherwise observable at the chest and extremity of the user, and the time at which it was detected. Accordingly, and as illustrated in graph 500, first adjustment amount 512 may be subtracted from first time 510 to produce aortic pulse time 515 and second adjustment amount 527 may be subtracted from second time 525 to produce peripheral pulse time 530. As further illustrated, first adjustment amount 512 and second adjustment amount 527 may be different amounts of time in order to account for different delays associated with each respective measurement system. While illustrated and described as subtracting time from a detection time of a pulse wave, embodiments described herein may alternatively make similar time adjustments to underlying measurements, such as light absorption data and/or radar data, prior to detecting the pulse wave from the underlying measurements, thereby resulting in an accurate detection time.

As further explained above, each pulse wave takes time to propagate outward from the heart to the extremities. The amount of time taken by the pulse wave to propagate from the heart to an extremity can be referred to as a PTT. For example, as illustrated in graph 500, time difference 540 measured from aortic pulse time 515 to peripheral pulse time 530 may represent the PTT of a pulse wave from a user's chest where aortic pulse wave 505 was detected to the user's extremity where peripheral pulse wave 520 was detected. Time difference 540 may also represent the phase difference between the continuous pulse waveform detected at the user's chest and the continuous pulse waveform detected at the user's extremity. In some embodiments, the phase difference between the pulse waveform detected at the user's chest and the pulse waveform detected at the user's extremity can be measured at a particular point in time and used to derive the PTT.

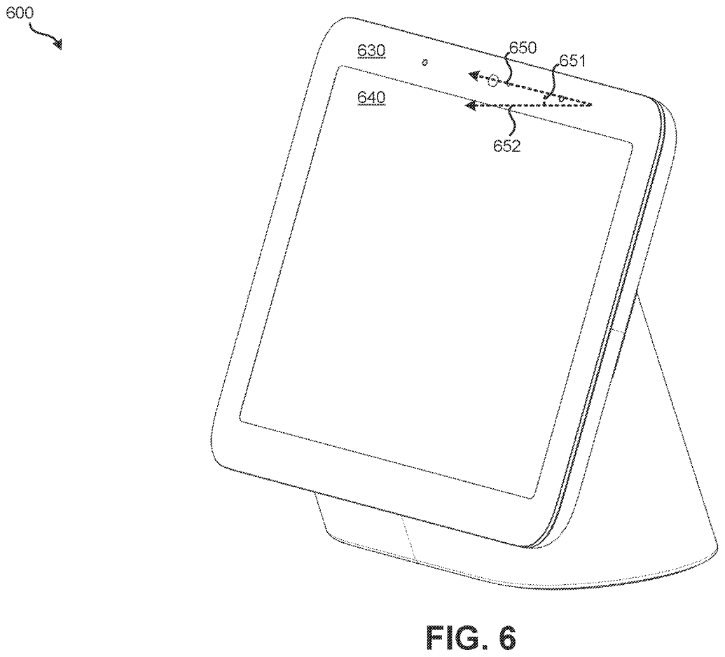

FIG. 6 illustrates an embodiment of a BP measurement device 600 (“device 600”). Device 600 can be a smart home hub device that can receive voice-based commands and provide an interface for a user to interact with various other smart home devices, view pictures, interact with media, and perform various other tasks. Device 600 may have a front surface that includes a front transparent screen 640 such that a display is visible. Such a display may be a touchscreen. Surrounding front transparent screen 640 may be an optically opaque region, referred to as bezel 630, through which radar subsystem 205 may have a field-of-view of the environment in front of device 600.

For purposes of the following description, the terms vertical and horizontal describe directions relative to a real world environment in general, with vertical referring to a direction aligned with acceleration due to gravity and horizontal referring to a direction perpendicular to vertical. Since the radar subsystem, which may be an Infineon® BGT60 radar chip, is roughly planar and is installed generally parallel to bezel 630 for spatial compactness of the device as a whole, and since the antennas within the radar chip lie in the plane of the chip, then, without beam targeting, a receive beam of radar subsystem 120 may be pointed in direction 650 that is generally normal to bezel 630. Due to a departure tilt of bezel 630 away from a purely vertical direction, which is provided in some embodiments to be about 25 degrees in order to facilitate easy user interaction with a touchscreen functionality of the transparent screen 640, direction 650 may point upwards from horizontal by departure angle 651. Assuming device 600 will typically be installed on a surface (e.g., table, counter, or nightstand) that is roughly the same height, or higher, than a user who will sit or lie down, it may be beneficial for the receive beam of radar subsystem 120 to be targeted in horizontal direction 652 or an approximately horizontal (e.g., between −5° and 5° from horizontal) direction. Therefore, vertical beam targeting can be used to compensate for departure angle 651 of the portion of device 600 in which radar subsystem 120 is present.

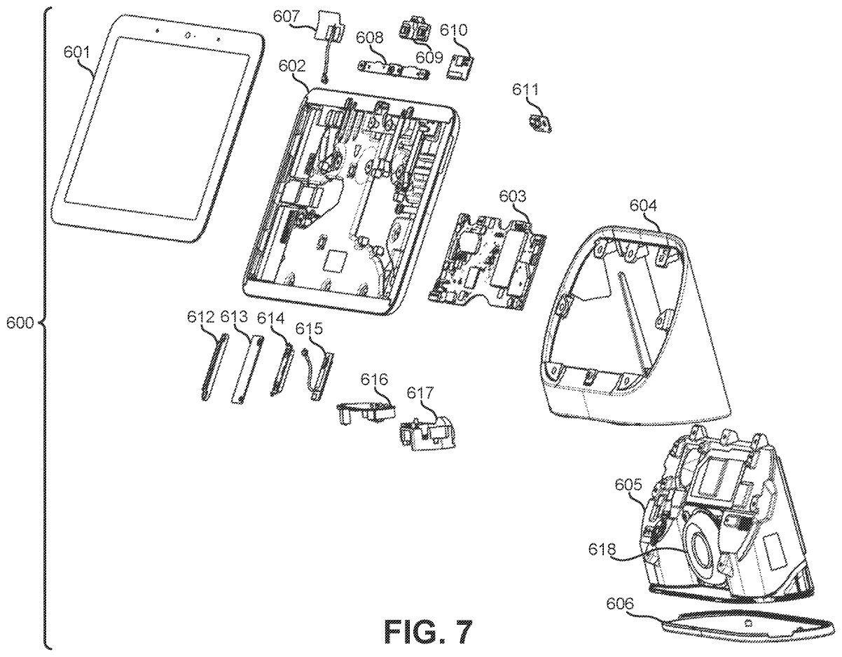

FIG. 7 illustrates an exploded view of an embodiment of BP measurement device 600. Device 600 can include: display assembly 601; display housing 602; main circuit board 603; neck assembly 604; speaker assembly 605; base plate 606; mesh network communication interface 607; top daughterboard 608; button assembly 609; radar assembly 610; microphone assembly 611; rocker switch bracket 612; rocker switch board 613; rocker switch button 614; Wi-Fi assembly 615; power board 616; and power bracket assembly 617. Device 600 can represent an embodiment of how BP measurement device 105 may be implemented.

Display assembly 601, display housing 602, neck assembly 604, and base plate 606 may collectively form a housing that houses all of the remaining components of device 600. Display assembly 601 may include an electronic display, which can be a touchscreen, that presents information to a user. Display assembly 601 may, therefore, include a display screen, which can include a metallic plate of the display that can serve as a grounding plane. Display assembly 601 may include transparent portions away from the metallic plate that allow various sensors a field of view in the general direction in which display assembly 601 is facing. Display assembly 601 may include an outer surface made of glass or transparent plastic that serves as part of the housing of device 600.

Display housing 602 may be a plastic or other rigid or semi-rigid material that serves as a housing for display assembly 601. Various components, such as main circuit board 603; mesh network communication interface 607; top daughterboard 608; button assembly 609; radar assembly 610; and microphone assembly 611 may be mounted on display housing 602. Mesh network communication interface 607; top daughterboard 608; radar assembly 610; and microphone assembly 611 may be connected to main circuit board 603, using flat wire assemblies. Display housing may be attached with display assembly 601, using an adhesive.

Mesh network communication interface 607 may include one or more antennas and may enable communication with a mesh network, such as a Thread-based mesh network. Wi-Fi assembly 615 may be located a distance from mesh network communication interface 607 to decrease the possibility of interference. Wi-Fi assembly 615 may enable communication with a Wi-Fi based network.

Radar assembly 610, which can include radar subsystem 120 or radar subsystem 205, may be positioned such that its RF emitter and RF receiver are away from the metallic plate of display assembly 601 and are located a significant distance from mesh network communication interface 607 and Wi-Fi assembly 615. These three components may be arranged in approximately a triangle to increase the distance between the components and decrease interference. For instance, in device 600, a distance of at least 74 mm between Wi-Fi assembly 615 and radar assembly 610 may be maintained. A distance of at least 98 mm between mesh network communication interface 607 and radar assembly 610 may be maintained. Additionally, distance between radar assembly 610 and speaker 618 may be desired to minimize the effect of vibrations on radar assembly 610 that may be generated by speaker 618. For instance, for device 600, a distance of at least 79 mm between radar assembly 610 and speaker 618 may be maintained. Additionally, distance between the microphones and radar assembly 610 may be desired to minimize any possible interference from the microphones on received radar signals. Top daughterboard 608 may include multiple microphones. For instance, at least 12 mm may be maintained between a closest microphone of top daughterboard 608 and radar assembly 610.

Other components may also be present. A third microphone assembly may be present, microphone assembly 611, which may be rear-facing. Microphone assembly 611 may function in concert with the microphones of top daughterboard 608 to isolate spoken commands from background noise. Power board 616 may convert power received from an AC power source to DC to power the components of device 600. Power board 616 may be mounted within device 600 using power bracket assembly 617. Rocker switch bracket 612, rocker switch board 613, and rocker switch button 614 may be collectively used to receive user input, such as up/down input. Such input may be used, for example, to adjust a volume of sound output through speaker 618. As another user input, button assembly 609 may include a toggle button that a user can actuate. Such a user input may be used to activate and deactivate all microphones, such as for when the user desires privacy and/or does not want device 600 to respond to voice commands.

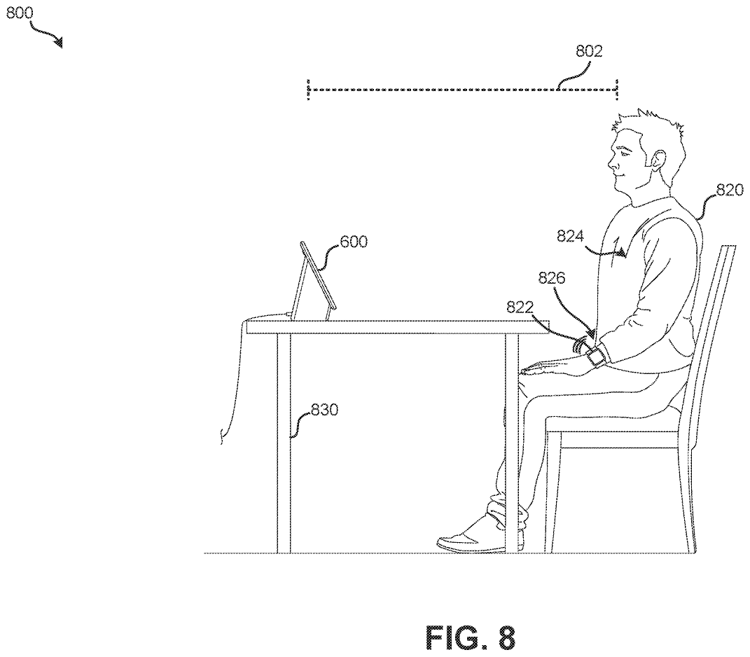

FIG. 8 illustrates an embodiment 800 of a user having the user's BP measured using a distributed BP measurement system. As illustrated, the distributed BP measurement system can include BP measurement device 600 and remote sensing device 822. BP measurement device 600, which can represent an embodiment of BP measurement device 105, can be stationary on a surface, such as table 830. Remote sensing device 822, which can represent an embodiment of remote sensing device 160, can be worn by, or otherwise in contact with, user 820 at or near extremity 826, such as a wrist, finger, ear, toe, ankle, and the like. As further described above, remote sensing device 822 may include a suite of sensors and/or sensor subsystems, such as optical subsystem 172, configured to detect and collect vital sign data from user 820, such as PPG data from extremity 826.

BP measurement device 600 and remote sensing device 822 may be in communication with each other via one or more wired or wireless communication protocols. For example, user 820 may pair remote sensing device 822 via Bluetooth® or a mesh network connection, such as Thread®. Additionally, or alternatively, BP measurement device 600 and remote sensing device 822 may communicate over a LAN, such as a Wi-Fi network. In some embodiments, BP measurement device 600 and/or remote sensing device 822 perform an authentication procedure before commencing communication with each other. For example, after detecting the presence of BP measurement device 600, remote sensing device 822 may request authentication from a cloud-based server system, such as cloud-based server system 190. The authentication request may include credentials for BP measurement device 600 and/or user account credentials for user 820. Based on the credentials for BP measurement device 600, cloud-based server system 190 may determine whether one or more preferences or permissions associated with a user account for user 820 allow for communication of vital sign data between remote sensing device 822 and BP measurement device 600. After authenticating BP measurement device 600 against the one or more preferences associated with the user account, BP measurement device 600 and/or remote sensing device 822 may proceed to initiate communication and sharing of vital sign data with each other.

Additionally, or alternatively, BP measurement device 600 may receive credentials for remote sensing device 822. Using the credentials for remote sensing device 822, BP measurement device 600 may proceed to identify a user account associated with remote sensing device 822 and/or verify that the user account has been associated with BP measurement device 600. Based on the identified and/or verified user account, BP measurement device 600 may determine whether user 820 has consented to have their BP measured and/or other health and vital sign data collected. After determining the BP of user 820 and/or collecting other health and vital sign data, BP measurement device may store the data in association with the user account (e.g., on BP measurement device 600 and/or on cloud-based server system 190).

After determining that user 820 wishes to have their BP measured, BP measurement device 600 and/or remote sensing device 822 may provide user 820 with one or more instructions (e.g., via electronic displays or speakers). For example, user 820 may be instructed to sit in a relaxed position with their legs and arms uncrossed. As another example, user 820 may be instructed to ensure that distance 802 between BP measurement device 600 and their aortic valve 824 meets predefined threshold distance and/or angle criteria, such as a minimum and/or maximum distance. In some embodiments, the predefined distance and/or angle criteria are based on the radar sensor capabilities of BP measurement device 600, as described above. In yet another example, user 820 may be instructed to ensure that remote sensing device 822 is in secure contact with the skin of user 820, located on extremity 826 at a predefined location, and/or at a predefined distance from aortic valve 824.

BP measurement device 600 and/or remote sensing device 822 may use data from one or more sensors to verify that the environment, condition, and/or position of user 820 meet one or more predefined criteria before proceeding to measure the BP of user 820. For example, radar-based object and/or motion detection may be used to determine that user 820 is within the range of the radar subsystem, that detected gross motion associated with user 820 is less than a predefined threshold, that there are no other living beings in the vicinity of user 820, and the like. As another example, IMU data from remote sensing device 822 may be used to detect the relative position of extremity 826. In yet another example, vital sign data, such as HR and/or RR data obtained from remote sensing device 822, may be used to determine whether user 820 is in a relaxed state by determining whether the vital sign data is within a threshold deviation of baseline vital sign measurements previously obtained for user 820.

Based on a determination that the one or more predefined criteria have not been met, BP measurement device 600 and/or remote sensing device 822 may provide additional instructions to user 820 to address the failing conditions. For example, user 820 may be instructed to move closer to BP measurement device 600. As another example, user 820 may be instructed to perform a controlled breathing exercise until they are determined to be in a resting state (e.g., based on subsequent HR and/or RR measurements).

As further described above, before commencing with the BP measurement, BP measurement device 600 and remote sensing device 822 may synchronize their respective clocks to ensure an accurate BP measurement. Additionally, or alternatively, BP measurement device 600 may adjust timestamps associated with either set of data based on a determined time difference (e.g., drift) between a clock of BP measurement device 600 and remote sensing device 822. Once synchronized, BP measurement device 600 may begin collecting, and/or analyzing radar data from its radar subsystem while receiving vital sign data (e.g., PPG data) from remote sensing device 822.

BP measurement device 600 may begin analyzing the collected and received data as soon as the BP measurement process has been initiated. BP measurement device 600 may continue to collect and analyze data until a BP measurement has been determined with a predefined accuracy or confidence. Additionally, or alternatively, BP measurement device 600 may collect and store the data for a predefined time period before bulk processing the collected data to determine a BP for user 820. For example, BP measurement device 600 may collect data for 15 seconds, 30 second, 1 minute, or a similarly suitable amount of time based on the amount of data required to generate a BP determination with an accuracy or confidence that meets predefined threshold criteria. Based on the data collected by BP measurement device 600 and remote sensing device 822, a BP measurement engine, such as BP measurement engine 230 and/or BP measurement engine 310, may proceed to determine a BP measurement for user 820.

After determining the BP measurement for user 820, BP measurement device 600 may output the BP measurement to user 820. For example, BP measurement device 600 may display the BP measurement on a display of BP measurement device 600 and/or cause a display of remote sensing device 822 to display the BP measurement. Additionally, or alternatively, BP measurement device 600 and/or remote sensing device 822 may store the BP measurement with prior BP measurements for user 820. The BP measurements may be stored locally (e.g., on BP measurement device 600) and/or remotely (e.g., within cloud-based server system 190) in association with an account of user 820 and/or device credentials for remote sensing device 822.

BP measurement device 600 and/or remote sensing device 822 may proceed to make additional determinations about the health of user 820 by analyzing the current BP measurement in the context of prior BP measurements for user 820 and/or anonymized BP measurement statistics for other users with matching demographics as user 820. For example, based on a determination that the current BP measurement is higher or lower than prior BP measurements for user 820, BP measurement device 600 may make a determination that user 820 might be suffering from hypertension or hypotension. As another example, by analyzing trends associated with current and prior BP measurements for user 820, BP measurement device 600 may determine that the BP of user 820 is higher or lower at different points in the day. Based on this determination, BP measurement device 600 and/or remote sensing device 822 may provide suggestions to user 820 to address the condition or seek further medical advice. Additionally, or alternatively, BP measurement device 600 may detect a trend in recent BP measurements for user 820 indicating that a BP calibration profile may need to be updated by taking the BP of user 820 with a traditional BP measurement device (e.g., a wrist or arm cuff) close in time to the distributed BP measurement system performing a subsequent BP measurement.

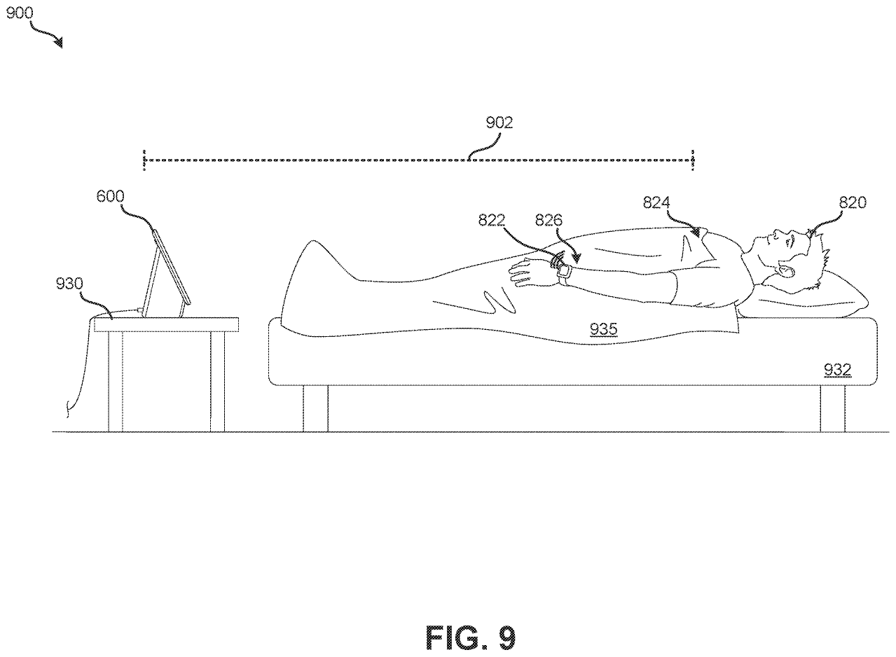

While described above as measuring the BP of user 820 while sitting down, other positions, orientations, and environments may be equally suitable for measuring the BP of user 820. FIG. 9 illustrates another embodiment 900 of user 820 having their BP measured using a distributed BP measurement system. As illustrated, BP measurement device 600 can be stationary on a surface, such as nightstand 930 while user 820 is lying down on a surface (e.g., bed 932) such that aortic value 824 of user 820 is at distance 902 from BP measurement device 600. While BP measurement device 600 is illustrated as being adjacent to the foot of bed 932, other positions and/or locations of BP measurement device 600 with respect to user 820 are possible. For example, nightstand 930 and BP measurement device 600 may be at a head of bed 932. Since blanket 935 remains motionless or near motionless during the BP measurement and allows RF to pass through without much loss, it may have a negligible effect on the BP measurement.

Measuring BP at night while users are most relaxed may be advantageous to obtaining accurate baseline BP measurements. Accurate and consistent baseline measurements may improve the accuracy of other BP measurements taken throughout the day and/or contribute to subsequent determinations. For example, an accurate baseline BP measured at night may improve determinations related to hypertension or hypotension and the like. Additionally, or alternatively, BP measurements taken while a user is sleeping may be combined with other vital sign data, such as HR, RR, and the like, to make various determinations about a user's health, such as the occurrence of sleep apnea.

Before measuring the BP of user 820 at night, BP measurement device 600 and/or remote sensing device 822 may first verify that user 820 has consented to such measurements. For example, BP measurement device 600 may determine that a preference associated with an account of user 820 indicates that user 820 has consented to their BP being measured at night without prompting from user 820. As another example, BP measurement device 600 and/or remote sensing device 822 may display a notification to user 820 before going to sleep requesting consent from the user to perform a BP measurement. After measuring the BP of user 820, BP measurement device 600 and/or remote sensing device 822 may wait until a determination has been made that user 820 is awake before presenting the results of the BP measurement. For example, remote sensing device 822 may wait until one or more predefined movement criteria have been met before displaying a notification to user 820.

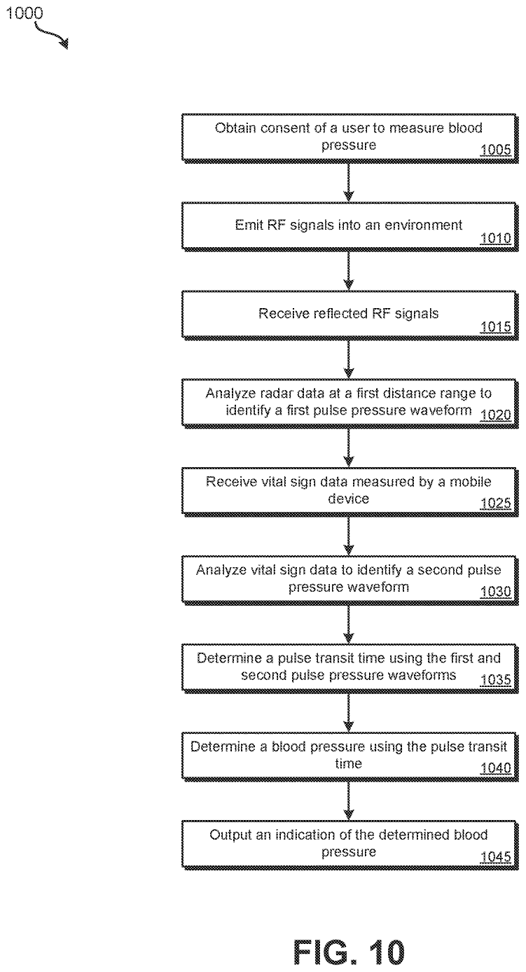

Various methods can be performed using the systems, devices, and arrangements of FIGS. 1-9. FIG. 10 illustrates an embodiment of a method 1000 for measuring BP using a distributed BP measurement system, such as detailed in relation to FIG. 1. The distributed BP measurement system used to perform method 1000 can include system 200. At block 1005, consent may be obtained to measure the blood pressure of the user. Block 1005 may include the user providing a vocal command requesting the user's blood pressure be measured or inputting such a command to a BP measurement device and/or remote sensing device, such as via a touchscreen. The consent may inform the user that their BP will be measured and, possibly, the resulting measurement stored in association with a user account of the user. For a first BP measurement, a user may be required to review and agree with a detailed end-user agreement. Method 1000 may only proceed if consent is provided by the user. Further, since method 1000 requires the user to remain in a particular physical position, consent can be inferred from the user assuming the physical position and remaining still in that position to allow the BP measurement to be made.

Method 1000 may optionally include outputting instructions using synthesized speech, via a message presented on a display of the BP measurement device, or both that instructs the user to be in a particular physical position. Such a position can involve the chest of the user being a set distance from the radar sensor of the BP measurement device. For example, the user may be instructed to sit or lie down between approximately 1 foot and 4 feet from the radar sensor. Additional or alternative instructions may instruct the user to ensure that optical sensors of the remote sensing device are in secure contact with the skin of an extremity of the user. For example, the user may be instructed to tighten a strap of a wrist or arm worn remote sensing device. Additionally, or alternatively, method 1000 may include synchronizing the clocks associated with the BP measurement device and the remote sensing device to a common time reference, as further described above.

At block 1010, RF signals may be emitted into an environment by the BP measurement device, such as by radar subsystem 205. The RF signals may be FMCW radar, as detailed in relation to FIG. 4. Reflections of the emitted RF signals may be received at block 1015. After receiving the reflected RF signals, initial processing may be performed on the reflected RF signals. For example, timestamps associated with the reflected RF signals may be adjusted to reflect processing delays. As another example, reflected RF signals may be analyzed to obtain distance-binned frequency measurements, as detailed in relation to radar processing module 210. Further detail regarding the processing of the received RF signals can be found in relation to FIG. 2.

At block 1020, the radar data may be analyzed at a first distance range by the BP measurement device. The first distance range can correspond to a distance bin at which the user's aortic valve is present. Since the aortic value moves a significant distance with each heartbeat compared to blood vessels, the distance bin at which a highest magnitude frequency component is present that corresponds to a resting heart rate (e.g., between 40 and 150 BPM) may be selected. Based on motion detected within the first distance range, a first pulse pressure waveform may be identified and stored. In some embodiments, block 1020 involves applying a trained machine learning model, such as a trained neural network, to the radar data to determine the first distance range and/or detect the first pulse pressure waveform.

At block 1025, vital sign data measured by a mobile device may be received. The mobile device may be a remote sensing device, as described in relation to FIG. 1. For example, the mobile device may include an optical subsystem, such as optical subsystem 172, configured to measure vital sign data, such as PPG data. The vital sign data may be measured by the mobile device at an extremity of the user, such as a wrist, finger, foot, ankle, or arm, of the user.

At block 1030, the vital sign data may be analyzed to identify a second pulse pressure waveform. For example, the BP measurement device may apply one or more signal processing algorithms to PPG data, such as light absorption waveforms, to identify the second pulse pressure waveform at the extremity of the user. In some embodiments, the vital sign data is preprocessed by the mobile device to identify the second pulse pressure waveform. In such instances, block 1030 may include extracting the pulse pressure waveform from the vital sign data received from the mobile device. Prior to identifying the second pulse pressure waveform, the vital sign data may be time adjusted to account for delays in the collection, processing, and/or transmitting the vital sign data. Alternatively, such time adjustments may be made to the second pulse pressure waveform after identification. In some embodiments, block 1030 involves applying a trained machine learning model, such as a trained neural network, to the vital sign data to identify the second pulse pressure waveform.

At block 1020 and block 1030, in addition to analyzing the radar data and vital sign data to identify the first pulse pressure waveform at or near the user's aortic valve, and the second pulse pressure waveform at the extremity of the user, the HR, RR, and/or the DPWA of the user may be measured based on the first pulse pressure waveform, second pulse pressure waveform, or both.

At block 1035, a PTT between the aortic valve and the extremity may be determined using the first and second pulse pressure waveforms. For example, an amount of time that elapses from when a first pulse wave is detected at the aortic value (e.g., from the first pulse pressure waveform) to when it is detected at the extremity (e.g., from the second pulse pressure waveform) can be determined. Additionally, or alternatively, a phase difference between the first and second pulse pressure waveforms may be calculated, from which the PTT may be determined.

The PPT, possibly along with the DPWA, RR, and/or HR, can be used to determine the BP (systolic and diastolic) of the user at block 1040. In some embodiments, a look-up table or algorithm is used that takes the PTT, DPWA, RR, and/or HR as inputs and outputs the BP. In other embodiments, a trained machine learning model can take the PPT, DPWA, RR, and/or HR as inputs and outputs the BP values. At block 1050, an indication of the determined blood pressure is output. The indication of the determined BP can be output via synthesized speech, via displays of the BP measurement device and/or mobile device, and/or output to a cloud-based server system (e.g., for storage or access by the user from another computerized device).

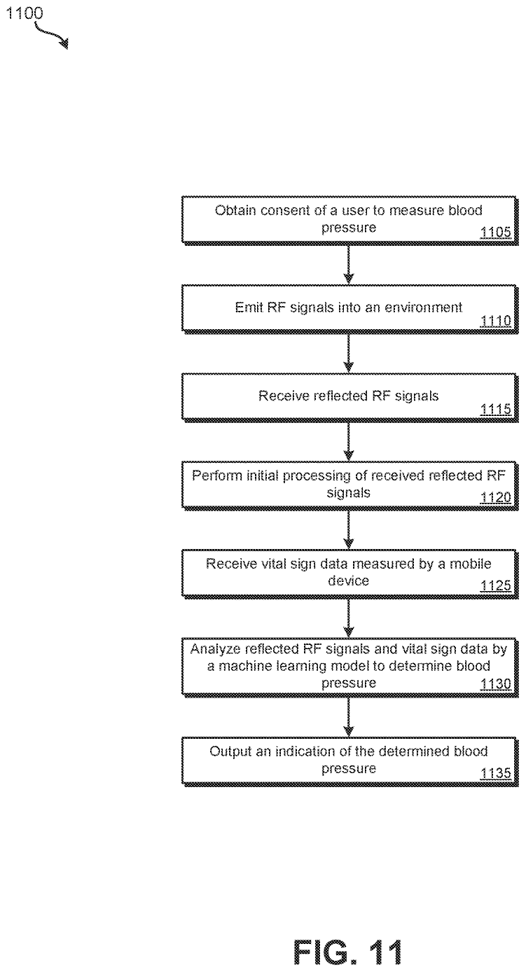

FIG. 11 illustrates an embodiment of method 1100 for measuring BP using a distributed BP measurement system, such as detailed in relation to FIG. 1. The distributed BP measurement system used to perform method 1100 can include system 300 of FIG. 3. At block 1105, consent may be obtained to measure the blood pressure of the user. Block 1105 may include the user providing a vocal command requesting the user's blood pressure be measured or inputting such a command to a BP measurement device or remote sensing device, such as via a touchscreen. The consent may inform the user that their BP will be measured and, possibly, the resulting measurement stored in association with a user account of the user. For a first BP measurement, a user may be required to review and agree with a detailed end-user agreement. Method 1100 may only proceed if consent is provided by the user. Further, since method 1100 requires the user to remain in a particular physical position, consent can be inferred from the user assuming the physical position and remaining still in that position to allow the BP measurement to be made.

Method 1100 may optionally include outputting instructions using synthesized speech, via a message presented on a display of the BP measurement device, or both that instructs the user to be in a particular physical position. Such a position can involve the chest of the user being a set distance from the radar sensor of the BP measurement device. For example, the user may be instructed to sit or lie down between approximately 1 foot and 4 feet from the radar sensor. Additional or alternative instructions may instruct the user to ensure that optical sensors of the remote sensing device are in secure contact with the skin of an extremity of the user. For example, the user may be instructed to tighten a strap of a wrist or arm worn remote sensing device. Additionally, or alternatively, method 1100 may include synchronizing the clocks associated with the BP measurement device and the remote sensing device to a common time reference, as further described above.

At block 1110, RF signals may be emitted into an environment by the BP measurement device, such as by radar subsystem 205. The RF signals may be FMCW radar, as detailed in relation to FIG. 4. Reflections of the emitted RF signals may be received at block 1115. At block 1120, initial processing of the received RF signals may be performed to obtain distance-binned frequency measurements and/or to adjust for various processing delays, as detailed in relation to radar processing module 210. Further detail regarding the processing of the received RF signals can be found detailed in relation to FIG. 2.

At block 1125, vital sign data measured by a mobile device may be received. The mobile device may be a remote sensing device, as described in relation to FIG. 1. For example, the mobile device may include an optical subsystem, such as optical subsystem 172, configured to measure vital sign data, such as PPG data. Additionally, or alternatively, the vital sign data may include pulse pressure waveform data identified by the mobile device. The vital sign data may be measured by the mobile device at an extremity of the user, such as a wrist, finger, foot, ankle, or arm, of the user. In some embodiments, after receiving the vital sign data, it is preprocessed to account for collection and/or processing delays, as detailed in relation to remote sensor module 225. Further detail regarding the processing of received vital sign data can be found in relation to FIG. 2.

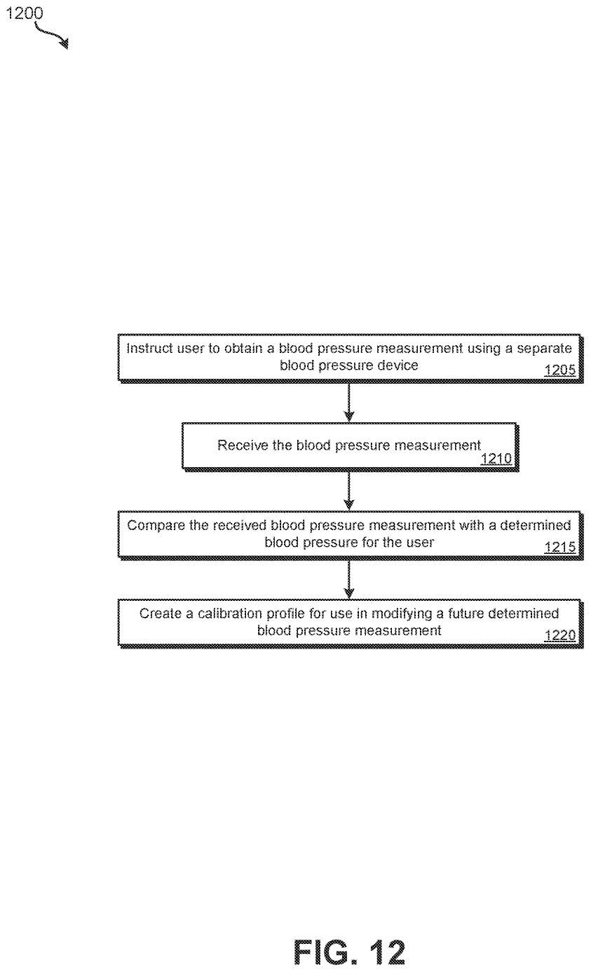

At block 1130, the radar data and vital sign data may be analyzed by a machine learning model. The machine learning model may be trained to use the radar data from the radar subsystem and the vital sign data from the optical subsystem as in input, and to output a blood pressure (systolic and diastolic) of the user. Therefore, the trained machine learning model directly analyzes the radar data and vital sign data to determine BP. In some embodiments, the trained machine learning model is further trained to use HR, RR, and/or DPWA for the user as inputs in determining the BP. Additionally, or alternatively, the trained machine learning model may automatically detect such features from the radar data and/or vital sign data, thereby reducing additional processing of the radar data and/or vital sign data prior to executing the trained machine learning model. The machine learning model may be a trained neural network. In some embodiments, an additional layer may be added or altered at the BP measurement device to factor in a calibration profile detailed in relation to FIG. 12.

At block 1135, an indication of the determined blood pressure is output. The indication of the determined BP can be output via synthesized speech, via displays of the BP measurement device and/or mobile device, and/or output to a cloud-based server system (e.g., for storage or access by the user from another computerized device).

To accurately determine BP using PTT, HR, RR, and/or DPWA, a calibration process may be completed for an individual user. The calibration process can account for the specific physical position the user will be in when the BP measurements are made using the distributed BP measurement system and/or characteristics particular to the user. FIG. 12 illustrates an embodiment of a method 1200 for calibrating a distributed BP measurement system. In method 1200, prior to using methods 1000 or 1100 to determine an accurate BP, the user may be instructed to use a separate blood pressure measurement device (that is expected to be accurate) to determine the user's BP at block 1205. The separate blood pressure device may use a mercury-gravity manometer, an aneroid gauge, or an electronic device. Such devices can use a cuff placed around an arm or wrist to measure blood pressure or a sensor placed on a finger. In other embodiments, a combination of an electrocardiogram (ECG) and finger or wrist PPG sensor may be used to determine PTT. The determined BP and/or PTT measurements may be input to the BP measurement device or to the cloud-based server system in communication with the BP measurement device via a computerized device (e.g., smartphone) at block 1210.

Prior to block 1205 or after block 1205 or block 1210, a user may then have their BP measured according to method 1000 or method 1100. Ideally, the two BP measurements are made within a short period of time of each other. For example, the user may perform one BP measurement and immediately perform the other, possibly while remaining seated or lying down. Whichever physical position the user uses, the user may be instructed to continue to use the same position for future BP measurements made by the BP measurement device.

At block 1215, the BP measured by the BP measurement device can be compared with the received BP measurement from block 1210. In some embodiments, the user is required or requested to perform multiple rounds of BP measurements using the BP measurement device and the separate BP measurement device to allow for a greater number of comparisons. The one or more comparisons performed at block 1215 are used to create a calibration profile at block 1220. In some embodiments, blood pressure measurements made via the separate blood pressure device and the corresponding BP measurements using the BP measurement device of FIG. 1 are used to create a training data set that can be used to optimize the machine learning models used in systems 200 and 300 or methods 1000 and 1100.

In some embodiments, the calibration profile can be simple offset values, an offset algorithm, or offset look-up that gets created. In other embodiments, a calibration model can be created, such as using one or few-shot learning, maximum likelihood estimation, or least-squares estimation.

The created calibration profile or calibration training data set can be used to adjust BP measurements made using the distributed BP measurement system, such as in method 1000 or method 1100. In method 1000, the calibration profile or calibration training data set may be used as part of block 1040 to adjust the BP measurement for the user. Referring to system 200, this calibration may be performed by BP determination engine 234. In method 1100, the calibration training data set may be used to create or adjust a layer of the machine learning model used at block 1130. Referring to system 300, analysis engine 311 may be modified based on the calibration profile.

It should be noted that the methods, systems, and devices discussed above are intended merely to be examples. It must be stressed that various embodiments may omit, substitute, or add various procedures or components as appropriate. For instance, it should be appreciated that, in alternative embodiments, the methods may be performed in an order different from that described, and that various steps may be added, omitted, or combined. Also, features described with respect to certain embodiments may be combined in various other embodiments. Different aspects and elements of the embodiments may be combined in a similar manner. Also, it should be emphasized that technology evolves and, thus, many of the elements are examples and should not be interpreted to limit the scope of the invention.

Specific details are given in the description to provide a thorough understanding of the embodiments. However, it will be understood by one of ordinary skill in the art that the embodiments may be practiced without these specific details. For example, well-known processes, structures, and techniques have been shown without unnecessary detail in order to avoid obscuring the embodiments. This description provides example embodiments only, and is not intended to limit the scope, applicability, or configuration of the invention. Rather, the preceding description of the embodiments will provide those skilled in the art with an enabling description for implementing embodiments of the invention. Various changes may be made in the function and arrangement of elements without departing from the spirit and scope of the invention.

Also, it is noted that the embodiments may be described as a process which is depicted as a flow diagram or block diagram. Although each may describe the operations as a sequential process, many of the operations can be performed in parallel or concurrently. In addition, the order of the operations may be rearranged. A process may have additional steps not included in the figure.

Having described several embodiments, it will be recognized by those of skill in the art that various modifications, alternative constructions, and equivalents may be used without departing from the spirit of the invention. For example, the above elements may merely be a component of a larger system, wherein other rules may take precedence over or otherwise modify the application of the invention. Also, a number of steps may be undertaken before, during, or after the above elements are considered. Accordingly, the above description should not be taken as limiting the scope of the invention.

1. A blood pressure measurement system, comprising:
a mobile device wearable by a user and configured to collect vital sign data at an extremity of the user; and
a stationary device in communication with the mobile device, the stationary device comprising:
a radio frequency (RF) emitter that emits RF signals;
an RF receiver that receives RF reflection signals based on the emitted RF signals being reflected; and
one or more processors configured to:
analyze the RF reflection signals at a first distance range to detect movement of the aortic valve of the user;
identify, based on the detected movement of the aortic valve, a first pulse pressure waveform at the aortic valve of the user;
analyze the vital sign data to identify a second pulse pressure waveform at the extremity of the user;
determine a pulse transit time (PTT) of a pulse pressure wave from the aortic valve of the user to the extremity of the user using the first pulse pressure waveform and the second pulse pressure waveform;
determine a blood pressure of the user based on the determined PTT; and
output an indication of the determined blood pressure.

2. The blood pressure measurement system of claim 1, wherein the vital sign data comprises photoplethysmography (PPG) data and the mobile device comprises an optical sensor that measures the PPG data at the extremity of the user.

3. The blood pressure measurement system of claim 1, wherein the stationary device further comprises: a microphone, a speaker, and an electronic display, wherein the indication of the determined blood pressure is output via the electronic display.

4. The blood pressure measurement system of claim 1, wherein the mobile device is a smart watch and the extremity of the user is a wrist of the user.

5. The blood pressure measurement system of claim 4, wherein the smart watch further comprises: a microphone, a speaker, and an electronic display, wherein the indication of the determined blood pressure is output via the electronic display.

6. The blood pressure measurement system of claim 1, wherein the RF reflection signals are associated with a first set of timestamps generated by a first clock of the stationary device, the vital sign data is associated with a second set of timestamps generated by a second clock of the mobile device, and the one or more processors are further configured to:
synchronize the first set of timestamps with the second set of timestamps; and
adjust the first set of timestamps, the second set of timestamps, or both based on a processing-delay difference between the stationary device and the mobile device.

7. The blood pressure measurement system of claim 1, wherein the one or more processors are further configured to:
receive an external blood pressure measurement made using a blood pressure device separate from the stationary device and the mobile device;
compare the external blood pressure measurement and the determined blood pressure measurement; and
create a calibration profile for use in modifying a future determined blood pressure measurement.

8. The blood pressure measurement system of claim 1, wherein the one or more processors are further configured to use the RF reflection signals and the vital sign data as inputs to a machine learning model trained to derive the blood pressure of the user based on the PTT.

9. A method for measuring blood pressure, comprising:
emitting, by a radar sensor of a stationary device, radio frequency (RF) signals;
receiving, by the radar sensor of the stationary device, RF reflection signals based on the emitted RF signals being reflected;
analyzing, by a processing system of the stationary device, the RF reflection signals at a first distance range to detect movement of the aortic valve of a user;
identify, based on the detected movement of the aortic valve, a first pulse pressure waveform at the aortic valve of the user;
receiving, by the processing system of the stationary device, vital sign data measured at an extremity of the user by a mobile device;
analyzing, by the processing system of the stationary device, the vital sign data to identify a second pulse pressure waveform at the extremity of the user;
determining, by the processing system of the stationary device, a pulse transit time (PTT) of a pulse pressure wave from the aortic valve of the user to the extremity of the user using the first pulse pressure waveform and the second pulse pressure waveform;
determining, by the processing system of the stationary device, a blood pressure of the user based on the determined PTT; and
outputting, by the processing system of the stationary device, an indication of the determined blood pressure.

10. The method for measuring blood pressure of claim 9, wherein the vital sign data comprises photoplethysmography (PPG) data, and the method further comprises:
measuring, by an optical sensor of the mobile device, the PPG data at the extremity of the user.

11. The method for measuring blood pressure of claim 9, wherein the RF reflection signals are associated with a first set of timestamps generated by a first clock of the stationary device, the vital sign data is associated with a second set of timestamps generated by a second clock of the mobile device, and the method further comprises:
synchronizing the first set of timestamps with the second set of timestamps.

12. The method for measuring blood pressure of claim 11, further comprising:
adjusting the first set of timestamps, the second set of timestamps, or both, based on a processing-delay difference between the stationary device and the mobile device.

13. The method for measuring blood pressure of claim 9, further comprising:
determining a phase difference between the first pulse pressure waveform and the second pulse pressure waveform, wherein the PTT is determined using the phase difference.

14. The method for measuring blood pressure of claim 9, wherein the extremity of the user is a wrist of the user.

15. The method for measuring blood pressure of claim 9, further comprising determining a heart rate based on analyzing the RF reflection signals, the second pulse pressure waveform, or both, wherein determining the blood pressure of the user is further based on the heart rate.

16. The method for measuring blood pressure of claim 9, further comprising determining a derived pulse waveform amplitude (DPWA) based on analyzing the RF reflection signals, wherein determining the blood pressure of the user is further based on the DPWA.

17. The method for measuring blood pressure of claim 9, further comprising determining a respiration rate based on analyzing the RF reflection signals, the second pulse pressure waveform, or both, wherein determining the blood pressure of the user is further based on the respiration rate.

18. The method for measuring blood pressure of claim 9, wherein analyzing the RF reflection signals at the first distance range comprises analyzing data from the RF reflection signals using a trained machine learning model.

19. The method for measuring blood pressure of claim 9, further comprising:
receiving, by the processing system of the stationary device, an external blood pressure measurement made using a blood pressure device separate from the stationary device and the mobile device;
comparing, by the processing system, the external blood pressure measurement and the determined blood pressure measurement; and
creating, by the processing system, a calibration profile for use in modifying a future determined blood pressure measurement.

20. A stationary blood pressure measurement device, comprising:
a radar subsystem, comprising:
a radio frequency (RF) emitter that emits RF signals;
an RF receiver that receives RF reflection signals based on the emitted RF signals being reflected; and
a processing system, comprising one or more processors, in communication with the radar subsystem, wherein the processing system is configured to:
analyze the RF reflection signals at a first distance range to detect movement of the aortic valve of a user;
identify, based on the detected movement of the aortic valve, a first pulse pressure waveform at the aortic valve of the user;
receive vital sign data measured at an extremity of the user by a mobile device;
analyze the vital sign data to identify a second pulse pressure waveform at the extremity of the user;
determine a pulse transit time (PTT) of a pulse pressure wave from the aortic valve of the user to the extremity of the user using the first pulse pressure waveform and the second pulse pressure waveform;
determine a blood pressure of the user based on the determined PTT; and
output an indication of the determined blood pressure.
